(19) United States
(12) Patent Application Publication
(10) Pub. No.: US 2011/0158980 A1
(43) Pub. Date: Jun. 30, 2011

(54) METHODS TO TREAT AND SCREEN FOR AGENTS TO TREAT OBESITY

(75) Inventors: Yaguang Si, Malden, MA (US); Kyongbum Lee, Winchester, MA (US)

(73) Assignee: TRUSTEES OF TUFTS COLLEGE, Medford, MA (US)

(21) Appl. No.: 13/056,349

(22) PCT Filed: Aug. 6, 2009
(86) PCT No.: PCT/US2009/052935

§ 371 Date: Mar. 15, 2011

Related U.S. Application Data
(60) Provisional application No. 61/088,104, filed on Aug. 12, 2008.

(51) Int. Cl. A61K 39/395; A61K 31/7088; A61K 38/02; A61K 31/713; A61K 31/192; A61K 31/185; C12N 5/077; C12Q 1/02; A61P 3/04

(52) U.S. Cl. 424/130.1; 514/44 A; 514/4.8; 514/570; 514/578; 435/375; 435/29

(57) ABSTRACT

Described herein are methods for treating obesity or promoting weight loss using agents that alter pyruvate flux in an adipocyte. Methods are provided for administering an agent or combination of agents to an obese individual to alter the flux of pyruvate in an adipocyte. Also described herein is a method for modifying triglyceride storage in an adipocyte by contacting an adipocyte with an agent or combination of agents that alter pyruvate flux.

### CROSS REFERENCE TO RELATED APPLICATION

This application claims benefit under 35 U.S.C. §119(e) of the U.S. Provisional Application No. 61/088,104, filed Aug. 12, 2008, the content of which is incorporated herein by reference in its entirety.

### GOVERNMENT SUPPORT

This invention was made with government support under DK067228 awarded by the National Institutes of Health. The government has certain rights in the invention.

### FIELD OF THE INVENTION

The invention relates to the treatment of obesity. The invention also relates to preventing triglyceride accumulation in adipocytes.

### BACKGROUND

Obesity has become one of the most pressing health concerns in the United States. As a chronic condition, obesity increases the risk of developing serious diseases and disorders, including type 2 diabetes, cardiovascular disease, hypertension and some forms of cancer (Willett, et al., 1999). It is estimated that 300,000 people die annually in the United States as a result of obesity (Kopelman, 2000).

White adipose tissue (WAT) has long been viewed as an inert organ, with the primary functions of storing excess energy as triglycerides (TG) and releasing fatty acids and glycerol to supply fuel substrates for other tissues during starvation. However, there is now a large body of evidence that implicates the WAT as an active player in the development of obesity and the progression of related metabolic disorders.

In the adipocyte, pyruvate is an important substrate for energy production, biosynthesis, and redox balance. Pyruvate is mainly formed from glucose through glycolysis. Its metabolic fate is determined by 3 major reactions: (a) efflux from the cell via reduction to lactate by lactate dehydrogenase (LDH); (b) complete oxidation or anaplerosis via decarboxylation to acetyl-CoA by the pyruvate dehydrogenase complex (PDC); and (c) anaplerosis via carboxylation to oxaloacetate (OAA) by pyruvate carboxylase (PC). The second and third reactions not only contribute to the production of ATP via the TCA cycle and oxidative phosphorylation, but also supply the carbon substrate (acetyl-CoA) for de novo fatty acid synthesis and NADPH generation (via the malate cycle) (Ballard F J, Hanson R W. J Lipid Res 1967; 8:73-9). The activities of both pyruvate carboxylase and PDC are low in undifferentiated 3T3-L1 cells, but increase 20- and 7-fold, respectively, upon differentiation (Freytag S O, Utter M F. Proc Natl Acad Sci USA 1980; 77:1321-5; Hu C W, Utter M F, Patel M S. J Biol Chem 1983; 258:2315-20). In genetically obese Zucker fatty rats, pyruvate carboxylase gene expression is elevated 2˜5-fold at the onset of obesity compared to lean controls (Lynch C J, McCall K M, Billingsley M L, et al. Am J Physiol 1992; 262:E608-18).

Previous studies have evaluated the role of pyruvate and pyruvate cycling in the setting of diabetes and insulin resistance. For example, Bahl et al (Bhal, J. J., et al Biochemical Pharmacology (1996) 53:67-74) indicates pyruvate carboxylase inhibitors may represent a potential new pharmacological approach to the treatment of hyperglycemia. However, this study did not suggest the treatment of obesity with a pyruvate carboxylase inhibitor. Similarly, prohibitin was shown to be an endogenous inhibitor of pyruvate carboxylase in adipocytes, which results in inhibition of insulin-stimulated glucose oxidation and fatty acid oxidation (Vessal, M., et al FEBS J (2006) 568-576). Prohibitin treatment manifests as a shift of metabolism from oxidative phosphorylation towards anaerobic glycolysis. However the results did not show that an opposing shift of metabolism from anaerobic glycolysis to citric acid cycle oxidation occurs with the use of a pyruvate carboxylase inhibitor.

### SUMMARY OF THE INVENTION

Described herein is a method for treating obesity, wherein an agent or combination of agents is administered to an obese individual to alter the flux of pyruvate in an adipocyte, such that the storage of triglyceride in the adipocyte is decreased. Also described herein is a method for modifying triglyceride storage in an adipocyte by contacting an adipocyte with an agent or combination of agents that alter pyruvate flux.

One aspect described herein relates to a method of treating obesity, comprising administering an agent that perturbs flux distribution of pyruvate in an individual with a BMI greater than about 30, wherein perturbing the flux of pyruvate decreases triglyceride storage in an adipocyte in the individual with BMI greater than 30.

In one embodiment of this aspect and all other aspects described herein, the agent shifts the flux of pyruvate from anaerobic glycolysis to the citric acid cycle.

In another embodiment of this aspect and all other aspects described herein, the agent is selected from the group consisting of: an RNA interference molecule, a small molecule, a protein, a polypeptide, an antibody, an antibody fragment or a metabolite analog.

In another embodiment of this aspect and all other aspects described herein, the agent inhibits at least one enzyme or enzyme complex selected from the group consisting of: lactate dehydrogenase, pyruvate dehydrogenase, pyruvate carboxylase, or any combination thereof.

In another embodiment of this aspect and all other aspects described herein, the agent comprises an inhibitor of pyruvate carboxylase.

In another embodiment of this aspect and all other aspects described herein, the small molecule is oxamate or phenylacetate.

In another embodiment of this aspect and all other aspects described herein, the RNA interference molecule comprises siRNA.

Another aspect described herein is a method of modifying triglyceride storage in an adipocyte, comprising contacting an adipocyte with an agent that perturbs flux of pyruvate, wherein the agent decreases storage of triglyceride in the adipocyte.

Another aspect described herein is a method of modifying triglyceride storage in an adipocyte, comprising contacting an adipocyte with at least one inhibitor of pyruvate carboxylase, wherein the inhibitor of pyruvate carboxylase decreases storage of triglyceride in an adipocyte.

Another aspect described herein is a method of reducing the number of adipocytes or preventing an increase in the number of adipocytes in an individual, comprising administering an agent that perturbs flux distribution of pyruvate in an individual undergoing a weight loss diet or to an individual initially having a BMI>30 who has reduced their body mass index by at least one point, wherein the agent permits the reduction of number of adipocytes or prevents an increase in the number of adipocytes in the individual.

Another aspect described herein relates to a method for screening a candidate agent that decreases triglyceride storage in a cell, the method comprising: (a) contacting a cell with ac candidate agent, and (b) assessing triglyceride levels in the cell, wherein if the triglyceride levels in the cell are lower than triglyceride levels in an untreated cell, then the candidate agent decreases storage of triglyceride in the adipocyte.

In one embodiment of this aspect, Oil Red O stain is used to assess triglyceride levels.

In another embodiment of this aspect, the cell comprises an adipocyte or an adipocyte cell line.

Also described herein is use of an agent that perturbs flux of pyruvate for treatment of obesity.

Another aspect described herein relates to the use of an agent that perturbs flux of pyruvate in the preparation of a medicament to treat obesity.

Also described herein is the use of an agent that perturbs flux of pyruvate for reducing triglyceride storage in an adipocyte.

### DEFINITIONS

As used herein the term “agent” refers to any organic or inorganic molecule, including modified and unmodified nucleic acids such as antisense nucleic acids, RNA interference agents such as siRNA or shRNA, peptides, peptidomimetics, receptors, ligands, vitamins, drugs, prodrugs, metabolite analogs, and antibodies.

The phrase “flux distribution of pyruvate” is used herein to describe the relative metabolic fate of pyruvate in an adipocyte. Pyruvate has three main metabolic fates in the adipocyte, which include (1) formation of lactate (via lactate dehydrogenase), (2) formation of acetyl CoA and entry into the TCA cycle (via the pyruvate dehydrogenase complex) and (3) formation of oxaloacetate for lipogenesis and TCA cycle activity (via pyruvate carboxylase). The fate of pyruvate is dependent on the metabolic status of the cell and its environment, for example the level of glucose that the cell is exposed to. The fate of pyruvate is also dependent on the level of oxygenation of the cell. For example, in the absence of oxygen (e.g., ischemia, hypoxia) pyruvate is converted to lactate in the cytosol. In the presence of oxygen, pyruvate can also be oxidized by the citric acid cycle. Therefore, the relative disposal of pyruvate through each of these three metabolic fates represents the “flux distribution of pyruvate”. Thus, if the flux is “shifted from e.g., anaerobic glycolysis to the citric acid cycle” by an agent, this means that a relatively higher amount of pyruvate is oxidized via the citric acid cycle than is utilized for the formation of lactate compared to untreated adipocytes.

As used herein, the phrase “shifts flux of pyruvate from anaerobic glycolysis to the citric acid cycle” refers to a decrease in the disposal of pyruvate through anaerobic glycolysis of at least 10% (by assessing e.g., the amount of lactate) in cells or subjects treated with a modulator of pyruvate flux compared to untreated cells or subjects. For example, the decrease is at least 20%, at least 30%, at least 40%, at least 50%, at least 60%, at least 70%, at least 80%, at least 90%, at least 95%, at least 99% or even 100% (i.e., absent) in the presence of a modulator of pyruvate flux.

The phrase “decreases triglyceride storage” is used herein to describe a net reduction in triglyceride accumulation due to a combination of (a) decreased synthesis, and/or (b) increased lipolysis. An agent is considered to “decrease triglyceride storage” when the triglyceride content of an isolated adipocyte (assessed by Oil Red O) staining is at least 5% lower than untreated adipocytes; preferably at least 10% lower, at least 20% lower, at least 30% lower, at least 40% lower, at least 50% lower, at least 60% lower, at least 70% lower, at least 80% lower, at least 90% lower or more in cells treated with an agent compared to untreated cells. It is important to note that a complete loss (e.g., 100%) of triglyceride accumulation in adipocytes is generally not desired. An agent that decreases triglyceride storage administered in vivo can be assessed for efficacy using any number of parameters including, for example weight loss, calculated BMI, percent weight loss, waist circumference, waist-to-hip ratio, fasting blood glucose level, serum triglyceride level, insulin level, high-density lipoprotein cholesterol level, low-density lipoprotein cholesterol level, systolic blood pressure, or diastolic blood pressure. For example, “decreases triglyceride storage” in these individuals is considered to be at least a 0.5 reduction in BMI following treatment with an agent; preferably at least a 1 point reduction, at least a 2 point reduction, at least a 5 point reduction, at least a 10 point reduction, at least a 15 point reduction, at least a 20 point reduction, at least a 25 point reduction or more, provided that the BMI of an individual does not go below 18.5, as this is considered underweight and can be associated with increased risk to the individual's health.

An “RNA interference molecule” as used herein, is defined as any agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), microRNA (miRNA) and small molecules which interfere with or inhibit expression of a target gene by RNA interference (RNAi).

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene, thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease will be, for example, at least 10%, at least 20%, at least 30%, at least 40%, at least 50%, at least 60%, at least 70%, at least 80%, at least 90%, at least 95% or at least 99% or more as compared to the expression of a target gene or the activity or level of the protein encoded by a target gene which has not been targeted by an RNA interference molecule. The terms “RNA interference” and “RNA interference molecule” as they are used herein are intended to encompass those forms of gene silencing mediated by double-stranded RNA, regardless of whether the RNA interfering agent comprises an siRNA, miRNA, shRNA or other double-stranded RNA molecule.

“Short interfering RNA” (siRNA), also referred to herein as “small interfering RNA” is defined as an RNA agent which functions to inhibit expression of a target gene, e.g., by RNAi. An siRNA may be chemically synthesized, may be produced by in vitro transcription, or may be produced within a host cell, for example, a genetically modified host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### BRIEF DESCRIPTION OF THE FIGURES

FIG. 1. shows a series of bar graphs depicting adipocyte proliferation and differentiation. FIG. 1A shows total DNA per well on day 12 post-induction. FIG. 1B shows gene expression levels of PPAR-γ and GPDH on day 8 post-induction, and FIG. 1C shows intracellular triglyceride (TG) on days 8, 10, 12 post-induction.

FIG. 2. shows a series of bar graphs depicting metabolite profile on day 12 post-induction; FIG. 2A shows intracellular free fatty acids (FFA), FIG. 2B shows FFA output, FIG. 2C shows glucose uptake, FIG. 2D shows lactate output, FIG. 2E shows glycerol output and FIG. 2F shows oxygen uptake rate (OUR).

FIG. 3. shows a representative metabolic schema showing flux distribution through major metabolic pathways in adipocytes on day 12 post-induction. Mitochondrial metabolites are shown in gray shaded boxes. Measured metabolites are indicated by bold italics.

### DETAILED DESCRIPTION

Described herein is a method for treating obesity, wherein an agent or combination of agents is administered to an obese individual to alter the flux of pyruvate in an adipocyte, such that the storage of triglyceride in the adipocyte is decreased as measured by e.g., a decrease in BMI of at least one point. Also described herein is a method for modifying triglyceride storage in an adipocyte by contacting an adipocyte with a combination of agents that alter pyruvate flux.

### Assessing Obesity

The methods described herein are useful in the treatment of obesity by administering an agent that perturbs pyruvate flux in an obese individual. For the purpose of this application, an individual is considered “obese” if the individual has a BMI greater than 30. In general, individuals with a BMI between 18.5 to 24.9 are considered to have a “healthy weight”, while individuals with a BMI ranging from 25.0 to 29.9 are considered to be “overweight”. A BMI less than 18.5 is considered underweight. Thus, the amount of an administered agent as defined herein can be titrated to achieve a BMI corresponding to “healthy weight” (e.g., 18.5-24.9). Body mass index is defined as the individual's body weight divided by the square of their height. For example, the BMI of an individual that is 5′9″ tall and weighs 202 lbs, is 29.9 and thus considered “overweight”. Accordingly, in one embodiment the overweight individual as intended in the methods of the invention has a BMI of 25.0-29.9. In one embodiment the obese individual as intended in the methods described herein has a BMI greater than or equal to 30.

Other parameters can also be measured to determine if an individual is obese. Waist circumference, for example, is a measure used to assess abdominal fat content. Women with a waist circumference greater than 35 inches and men with a waist circumference greater than 40 inches are considered to be at risk for obesity-related diseases. Waist circumference is assessed by measuring the distance around the individual's waist. Therefore, in one embodiment, the obesity in an individual is determined by measuring waist circumference and an individual is determined to be obese if the waist circumference is greater than 35.

In general, a waist-to-hip ratio greater than 1.0 in both sexes is considered to be a risk for obesity related disease, while a ratio of 0.9 for men, and 0.8 for women is generally considered healthy. Waist to hip ratio is determined by measuring the waist and hip circumference of the individual and dividing the value obtained for the waist measurement by the value obtained for the hip measurement. Therefore, in one embodiment, the obesity of an individual is determined by measuring the waist-to-hip ratio and an individual is determined to be obese if the waist-to-hip ratio is greater than 0.8.

Body fat percentage (also referred to herein as % body fat), can also be used to assess the obesity status of an individual. An individual's body fat percentage is the total weight of the individual's fat divided by the total body weight. An essential amount of body fat in women is 10-13% and in men is 2-5%. The recommended body fat percentage in women is 20-25%, while 30% or more is considered obese. In men, the recommended body fat percentage is 8-14%, while 25% or more is considered obese. Body fat percentage can be measured by a number of techniques including, but not limited to, the following: near-infrared interactance, dual energy X-ray absorptiometry, body average density measurement, bioelectrical impedance analysis, anthropometric methods, (e.g., Durnin-Womersley method), skinfold methods, as well as height and circumference methods. These methods are well within the abilities of one skilled in the art for use in assessing whether an individual is considered overweight or obese. In one embodiment, the overweight individual as intended in the methods of the invention has a body fat percentage between 26-29% in women, and 15-24% in men. In one embodiment, the obese individual as intended for the methods described herein has a body fat percentage of greater than 29% in women and greater than 24% in men. Therefore, in one embodiment, the obesity of an individual is determined by measuring body fat percentage and an individual is determined to be obese if the percent body fat is greater than 29% in women and 24% in men.

It is important to note that the methods described herein are useful in the reduction of e.g., BMI in an obese individual who may or may not have a concurrent obesity-related disease (e.g., insulin resistance). To be clear, the methods described herein are contemplated for use in the management of the state of obesity, rather than the reduction in an obesity related disease symptom. Thus, the methods described herein are useful in any obese or overweight individual and are not dependent on the presence of further metabolic disease. This distinction should be noted, since not all obese individuals will develop e.g., insulin resistance, however it is contemplated that any obese individual will benefit from the practice of the methods described herein.

### Flux Distribution of Pyruvate

It is shown herein that lipid accumulation can be impaired by inhibiting the entry of pyruvate into the mitochondria or lowering its utilization efficiency by the oxidation pathways (e.g., the citric acid cycle). In the adipocyte, one molecule of glucose is converted to two molecules of pyruvate through a series of enzymatic reactions known as “glycolysis”. Pyruvate formed in this manner has three distinct fates: (1) conversion to lactate (by lactate dehydrogenase), (2) conversion to acetyl CoA (by the pyruvate dehydrogenase complex), or (3) conversion to oxaloacetate (by pyruvate carboxylase). Conversion of pyruvate to lactate occurs under anaerobic conditions or when entry of pyruvate into the mitochondria is inhibited, while conversion of pyruvate to acetyl CoA permits entry into the oxygen dependent citric acid cycle, and conversion of pyruvate to oxaloacetate permits triglyceride accumulation in the adipocyte. Inhibition of pyruvate carboxylase prevents the regeneration of oxaloacetate by pyruvate, in a manner such that oxaloacetate is no longer available as a precursor for lipogenesis. A decrease in lipogenesis has the net effect of decreasing triglyceride accumulation in the adipocyte.

The “flux distribution of pyruvate” is a measure of the relative proportion of pyruvate that is converted into each of the three metabolic fates of pyruvate. This flux distribution is dynamic and therefore depends on the metabolic status of the cell and factors in the circulation. An agent can be administered to alter the “flux distribution of pyruvate” in an individual such that the relative proportion of e.g., lactate production is reduced as compared to the proportion in an untreated individual (referred to herein as “shifts” to the flux distribution of pyruvate). In general, shifting the flux distribution of pyruvate permits the metabolic status of the cell to be controlled, such that the net effect is to reduce triglyceride accumulation.

### Agents

A variety of different pharmaceutical/therapeutic agents can be used in conjunction with the methods described herein and include, but are not limited to, small molecules, proteins, antibodies, peptides and nucleic acids. In general, agents useful in the methods described herein will alter the metabolic status of the cell and in some cases will act directly on at least one of the pyruvate metabolic pathways. For example, small molecule inhibitors such as oxamate or phenylacetate can be used to alter pyruvate metabolism. Oxamate can be used to inhibit flux through lactate dehydrogenase, while phenylacetate inhibits flux through the pyruvate dehydrogenase complex and pyruvate carboxylase.

In one embodiment the agent inhibits pyruvate carboxylase or a variant thereof.

In another embodiment, the agent inhibits lactate dehydrogenase or a variant thereof.

In another embodiment, the agent inhibits at least one of the enzymes in the pyruvate dehydrogenase complex (e.g., pyruvate dehydrogenase, dihydrolipoyl transacetylase, or dihydrolipoyl dehydrogenase).

Metabolite mimetic inhibitors (e.g., inhibitors of pyruvate) can also be used in accordance with the present invention to decrease triglyceride storage. The design of inhibitor mimetics is known to those skilled in the art. In some cases an inhibitor mimetic can be a peptide or other relatively small molecule that has an activity that is the same or similar to that of a larger molecule, on which they are modeled. For example, a mimetic inhibitor can be designed such that it binds in the pyruvate binding pocket of e.g., pyruvate carboxylase and effectively inhibits pyruvate flux through that enzyme.

Variations and modifications to protein agents can be used to alter pyruvate flux distribution, and to provide means for targeting.

If so desired, more than one agent can be administered to an obese individual, wherein the agents inhibit at least one pathway or a combination of metabolic pathways of pyruvate.

siRNA agents

In one embodiment, the agent comprises an siRNA molecule that alters the flux distribution of pyruvate and can be directed against e.g., pyruvate carboxylase, pyruvate dehydrogenase complex, lactate dehydrogenase or any combination thereof. The genomic or mRNA sequences of each of these enzymes can be used to design an appropriate siRNA sequence as discussed below. For example, the following GenBank accession numbers are non-limiting examples of sequences for pyruvate carboxylase mRNA, untranslated regions, cDNA, or variants thereof: NM_{—}001040716, NM_{—}000920, NM_{—}022172, BC011617, U30890, U30889, U04641, U30891, K02282 and M26122, any of which can form the basis for siRNA sequence design.

Similarly, the following GenBank accession numbers are non-limiting examples of sequences for lactate dehydrogenase mRNA, untranslated regions, cDNA, or variants thereof: NM_{—}005566, AK312328, NM_{—}002300, NM_{—}017448, NM_{—}002301, NM_{—}194436, NM_{—}153486, AB209231, X13800, X13799, X13798, X13796, X13795, X03082, X03081, X03078, X03077, X03080, X03079, D28368, BT019765, BT019764, AY581313, AY286300, AF401097, AF401096, AF401095, AF401094, AY009108, M24514, M24512, M24511, M24510, Y00711, or X02152. It is important to note that there are multiple isoforms of lactate dehydrogenase, any of which can be used for the design of siRNA molecules. For the practice of the methods described herein it is preferred that the lactate dehydrogenase isoform targeted by siRNA is the isoform that contributes the most catalytic activity in the adipocyte of the obese individual being treated.

The following GenBank accession numbers are non-limiting examples of sequences for any of the enzymes that form the pyruvate dehydrogenase complex (including pyruvate dehydrogenase, dihydrolipoyl transacetylase, dihydrolipoyl dehydrogenase, or pyruvate dehydrogenase phosphatase) mRNA, untranslated regions, cDNA, or variants thereof: NM_{—}017990, NM_{—}000925, NM_{—}000108, NM_{—}001931, AK313872, NM_{—}000284, NM_{—}003477, EF444972, NM_{—}020786, NM_{—}005390, D90086, D90084, AK225621, AK225569, EF576990, Y00978, J03576, AF317200, L48690, M21447, AF155661, AF085747, U23825, U75940, U75939, U75938, U75937, U75936, U75935, U75934, U75933, X57778, AH002929, M27257, M29163, M29161, M29162, M29160, M29159, M29158, M29157, M29156, M29155, L13318, M34055, M86808, J03503, M34056, M54788, AH002930, M19124, M19123, M29141, J03575, M24848, or M34479. It is important to note that the pyruvate dehydrogenase complex comprises three enzymes (pyruvate dehydrogenase, dihydrolipoyl transacetylase, and dihydrolipoyl dehydrogenase) which are necessary for catalytic conversion of pyruvate to acetyl CoA and further comprises two regulatory enzymes (pyruvate dehydrogenase kinase, and pyruvate dehydrogenase phosphatase). It is known in the art that phosphorylation of the E1 subunit of the pyruvate dehydrogenase complex (PDC) by pyruvate dehydrogenase kinase inhibits the catalytic activity of the complex, while conversely de-phosphorylation of the complex by the PDC phosphatase restores PDC activity. Therefore, in addition to inhibiting the expression of any of the enzymes in the PDC complex with an siRNA molecule, it is also contemplated herein that the PDC phosphatase can also be repressed using siRNA techniques. The loss of PDC phosphatase activity would permit the PDC complex to remain in its phosphorylated and inactive state.

In one embodiment, the siRNA is obtained from a commercial source such as INVITROGEN™, AMBION™, or DHARMACON™, among others. In one embodiment, siRNAS useful in the methods described herein are commercially obtained from AMBION™ and include, but are not limited to, catalog numbers s10088, s10089, and s10090.

In another embodiment, an siRNA sequence targeting pyruvate carboxylase, pyruvate dehydrogenase complex or lactate dehydrogenase can be designed from the nucleotide sequence of the desired enzyme (e.g., pyruvate carboxylase, lactate dehydrogenase or PDC) using an siRNA predictive tool such as, for example, the Whitehead Institute siRNA Selection program (available on the world wide web at jura.wi.mit.edu/bioc/siRNAext/), AMBION™ siRNA target finder (available on the world wide web at ambion.com/techlib/misc/siRNA_finder.html), DHARMACON™ SIDESIGN® center (available on the world wide web at dharmacon.com/DesignCenter/DesignCenterPage.aspx), GenScript siRNA Target Finder (available on the world wide web at genscript.com/ssl-bin/app/rnai), Molecula (available on the world wide web at molecula.com/new/siRNA_inquiry.html), among others.

In another embodiment, the siRNA sequence can be designed based on the nucleotide sequence of the target enzyme according to various guidelines, including but not limited to the guidelines presented in the following publications: Elbashir et al (2001) Genes Dev.; 15(2):188-200, Schwarz D S, et al (2003) Cell; 115(2):199-208, Khvorova A, et al (2003) Cell; 115(2):209-16, Pi Y., et al (2006) Nat Methods; 3(9):670-6, Reynolds, A., et al (2004) Nat Biotechnol; 22(3):326-30, Hsieh A C., et al (2004) Nucleic Acids Res; 32(3):893-901, or Ui-Tei K., et al (2004) Nucleic Acids Res 32(3):936-48, which are incorporated herein by reference in their entirety.

The skilled person understands that double-stranded oligonucleotides comprising a duplex structure of between 20 and 23, but specifically 21, base pairs have been hailed as particularly effective in inducing RNA interference (Elbashir et al., EMBO 2001, 20:6877-6888). However, others have found that shorter or longer double-stranded oligonucleotides can be effective as well.

Double-stranded and single-stranded oligonucleotides that are effective in inducing RNA interference are also referred to as siRNA, RNAi agent, or iRNA agent, herein. These RNA interference inducing oligonucleotides associate with a cytoplasmic multi-protein complex known as RNAi-induced silencing complex (RISC). In many embodiments, single-stranded and double-stranded RNAi agents are sufficiently long that they can be cleaved by an endogenous molecule, e.g. by Dicer, to produce smaller oligonucleotides that can enter the RISC machinery and participate in RISC mediated cleavage of a target sequence, e.g. a target mRNA.

In the context of this invention, the term “oligonucleotide” refers to a polymer or oligomer of nucleotide or nucleoside monomers consisting of naturally occurring bases, sugars and intersugar (backbone) linkages. The term “oligonucleotide” also includes polymers or oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases

The RNAi agents may comprise any oligonucleotide modification known to one of skill in the art. In certain instances, it may be desirable to modify one or both strands of a double-stranded oligonucleotide. In some cases, the two strands will include different modifications. In other instances, multiple different modifications can be included on each of the strands. The various modifications on a given strand may differ from each other, and may also differ from the various modifications on other strands. For example, one strand may have a modification, e.g., a modification described herein, and a different strand may have a different modification, e.g., a different modification described herein. In other cases, one strand may have two or more different modifications, and the another strand may include a modification that differs from the at least two modifications on the first strand.

### Double-Stranded RNAi Agents

Double-stranded RNAi agents comprise two oligonucleotide strands that are sufficiently complementary to hybridize to form a duplex structure. Typically, the duplex structure is between 15 and 30, between 18 and 25, between 19 and 24, or between 19 and 21 base pairs in length. In certain embodiments, longer double-stranded oligonucleotides of between 25 and 30 base pairs in length are preferred. In certain embodiments, shorter double-stranded oligonucleotides of between 10 and 15 base pairs in length are preferred. In another embodiment, the double-stranded oligonucleotide is at least 21 nucleotides long.

In one embodiment, the double-stranded RNAi agent comprises a sense strand and an antisense strand, wherein the antisense RNA strand has a region of complementarity which is complementary to at least a part of a target sequence, and the duplex region is 14-30 nucleotides in length. Similarly, the region of complementarity to the target sequence is between 14 and 30, more generally between 18 and 25, yet more generally between 19 and 24, and most generally between 19 and 21 nucleotides in length.

In many embodiments, the double-stranded RNAi agent is sufficiently large that it can be cleaved by an endogenous molecule, e.g., by Dicer, to produce smaller double-stranded RNAi agents. In one embodiment, the double-stranded RNAi agent modulates the expression of a target gene via RISC mediated cleavage of the target sequence.

In certain embodiments, the double-stranded region of a double-stranded RNAi agent is equal to or at least, 10, 11, 12, 13, 14, 15, 16, 17, 18, 19, 20, 21, 22, 23, 23, 24, 25, 26, 27, 28, 29, or 30 nucleotide pairs in length.

In certain embodiments, the antisense strand of a double-stranded RNAi agent is equal to or at least 14, 15, 16, 17, 18, 19, 20, 21, 22, 23, 23, 24, 25, 26, 27, 28, 29, or 30 nucleotides in length.

In certain embodiments, the sense strand of a double-stranded RNAi agent is equal to or at least 10, 11, 12, 13, 14, 15, 16, 17, 18, 19, 20, 21, 22, 23, 23, 24, 25, 26, 27, 28, 29, or 30 nucleotides in length.

In certain embodiments, one strand has at least one stretch of 1-5 single-stranded nucleotides in the double-stranded region. In certain other embodiments, both strands have at least one stretch of 1-5 single-stranded nucleotides in the double stranded region. When both strands have a stretch of 1-5 single-stranded nucleotides in the double stranded region, such single-stranded nucleotides may be opposite to each other or they can be located such that the second strand has no single-stranded nucleotides opposite to the single-stranded oligonucleotides of the first strand and vice versa.

The double-stranded RNAi agents may have a single strand overhang or terminal unpaired region, at the one or both ends of the duplex region. In certain embodiments, the overhangs are 1-4, more generally 2-3 nucleotides in length. In one embodiments, the antisense strand comprises a 2 nucleotide single strand overhang on the 3′ end. In another embodiment, the double-stranded RNAi agent comprises 2 nucleotide single strand overhang on the 3′ end of the sense strand and on the 3′ end of the antisense strand.

In certain embodiments, the two strands of a double-stranded RNAi agent are linked together. The two strands be linked to each other at both ends, or at one end only. The two strands can be linked together by an oligonucleotide linker including, but not limited to, (N)n; wherein N is independently a modified or unmodified nucleotide and n is 3-10. The two strands can also be linked together by a non-nucleosidic linker, e.g. a linker described herein. It will be appreciated by one of skill in the art that any oligonucleotide chemical modifications or variation known in the art may be used in the oligonucleotide linker.

Double stranded RNAi agents, where the two strands are linked together, will have a duplex region equal to or at least 14, 15, 15, 16, 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length.

The hairpin RNAi agents may have a single strand overhang or terminal unpaired region, in some embodiments at the 3′, and in certain embodiments on the antisense side of the hairpin. In certain embodiments, the overhangs are 1-4, more generally 2-3 nucleotides in length.

### Single-Stranded RNAi Agents

A “single-stranded RNAi agent” as used herein, is an RNAi agent which is made up of a single molecule. A single-stranded RNAi agent may include a duplexed region, formed by intra-strand pairing, e.g., it may be, or include, a hairpin or pan-handle structure. Single-stranded RNAi agents may be antisense with regard to the target molecule. A single-stranded RNAi agent may be sufficiently long that it can enter the RISC and participate in RISC mediated cleavage of a target mRNA.

The single-stranded RNAi agents comprise nucleotide sequence that is substantially complementary to a “sense” nucleic acid encoding a gene expression product, e.g., complementary to the coding strand of a double-stranded cDNA molecule or complementary to an RNA sequence, e.g., a pre-mRNA, mRNA, miRNA, or pre-miRNA. The region of complementarity is less than 30 nucleotides in length, and at least 15 nucleotides in length. Generally, the single stranded RNAi agents are 10 to 25 nucleotides in length (e.g., 11, 12, 13, 14, 15, 16, 18, 19, 20, 21, 22, 23, or 24 nucleotides in length). In one embodiment the single stranded RNAi agent is 25-30 nucleotides. In one embodiment, the single-stranded oligonucleotide is 15-29 nucleotides in length. In certain embodiments, the single-stranded RNAi agent is at least 29, at least 35, at least 40, or at least 50 nucleotides in length. In certain embodiments, it is less than 200, 100, or 60 nucleotides in length. In certain embodiments single-stranded RNAi agents are 5′ phosphorylated or include a phosphoryl analog at the 5′ prime terminus. Single RNAi agents having less than 100% complementarity to the target mRNA, RNA or DNA are also embraced by the present invention.

Given the combination of the nucleotide sequences for the target enzymes, the online siRNA sequence predictive software, and the aforementioned guidelines, it is entirely possible for one of skill in the art to be able to design an siRNA sequence for the purposes of the methods described herein. The predicted siRNA sequences can be tested in silico by performing a BLAST search of a human genomic database using the chosen predicted siRNA sequence. Preferred siRNA sequences have high sequence homology to the target enzyme sequence and have limited cross-reactions with other nucleotide sequences. It is well within the abilities of one skilled in the art to choose an appropriate siRNA sequence with a high probability of success in the methods described herein using this approach.

The siRNA preferably targets only one sequence. Each of the RNA interfering agents, such as siRNAs, can also be screened for potential off-target effects by, for example, expression profiling. Such methods are known to one skilled in the art and are described, for example, in Jackson et al. Nature Biotechnology 6:635-637, 2003. In addition to expression profiling, one may also screen the potential target sequences for similar sequences in the sequence databases to identify potential sequences which may have off-target effects. For example, according to Jackson et al. (Id.) 15, or perhaps as few as 11 contiguous nucleotides, of sequence identity are sufficient to direct silencing of non-targeted transcripts. Therefore, one may initially screen the proposed siRNAs to avoid potential off-target silencing using the sequence identity analysis by any known sequence comparison methods, such as BLAST.

siRNA sequences are chosen to maximize the uptake of the antisense (guide) strand of the siRNA into RISC and thereby maximize the ability of RISC to target human GGT mRNA for degradation. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC and direct the sequence-specific cleavage of e.g., the human pyruvate carboxylase mRNA.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

In a preferred embodiment, the siRNA or modified siRNA is delivered or administered in a pharmaceutically acceptable carrier. Additional carrier agents, such as liposomes, can be added to the pharmaceutically acceptable carrier.

In another embodiment, the siRNA is delivered by delivering a vector encoding small hairpin RNA (shRNA) in a pharmaceutically acceptable carrier to the cells in an organ of an individual. The shRNA is converted by the cells after transcription into siRNA capable of targeting, for example, pyruvate carboxylase. In one embodiment, the vector is a regulatable vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents.

One method to deliver the siRNAs is catheterization of the blood supply vessel of the target organ.

Other strategies for delivery of the RNA interfering agents, e.g., the siRNAs or shRNAs used in the methods of the invention, may also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

The RNA interfering agents, e.g., the siRNAs targeting pyruvate carboxylase mRNA, may be delivered singly, or in combination with other RNA interfering agents, e.g., siRNAs, such as, for example siRNAs directed to other cellular genes. Pyruvate carboxylase siRNAs may also be administered in combination with other pharmaceutical agents which are used to treat or prevent triglyceride accumulation in the adipocyte or obesity.

Methods of delivering RNA interfering agents, e.g., an siRNA, or vectors containing an RNA interfering agent, to the target cells, e.g., adipocytes or other desired target cells, for uptake include injection of a composition containing the RNA interfering agent, e.g., an siRNA, or directly contacting the cell, e.g., an adipocyte, with a composition comprising an RNA interfering agent, e.g., an siRNA. In another embodiment, RNA interfering agents, e.g., an siRNA may be injected directly into any blood vessel, such as vein, artery, venule or arteriole, via, e.g., hydrodynamic injection or catheterization. Administration may be by a single injection or by two or more injections. The RNA interfering agent is delivered in a pharmaceutically acceptable carrier. One or more RNA interfering agents may be used simultaneously.

In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNA interference effectively into cells. For example, an antibody-protamine fusion protein when mixed with siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein.

A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501).

The RNA interfering agents, e.g., the siRNAs or shRNAs, can be introduced along with components that perform one or more of the following activities: enhance uptake of the RNA interfering agents, e.g., siRNA, by the cell, e.g., adipocytes, inhibit annealing of single strands, stabilize single strands, or otherwise facilitate delivery to the target cell and increase inhibition of the target gene, e.g., pyruvate carboxylase.

### In Vivo Delivery of RNA Interference (RNAi) Molecules

In general, any method of delivering a nucleic acid molecule can be adapted for use with an RNAi interference molecule (see e.g., Akhtar S, and Julian R L. (1992) Trends Cell. Biol. 2(5):139-144; WO94/02595, which are incorporated herein by reference in their entirety). There are three factors that are important to consider to successfully deliver an RNAi molecule in vivo: (a) biological stability of the RNAi molecule, (2) preventing non-specific effects, and (3) accumulation of the RNAi molecule in the target tissue. The non-specific effects of an RNAi molecule can be minimized by local administration by e.g., direct injection into a tissue including, for example, a tumor or topically administering the molecule.

Local administration of an RNAi molecule to a treatment site limits the exposure of the e.g., siRNA to systemic tissues and permits a lower dose of the RNAi molecule to be administered. Several studies have shown successful knockdown of gene products when an RNAi molecule is administered locally. For example, intraocular delivery of a VEGF siRNA by intravitreal injection in cynomolgus monkeys (Tolentino, M J., et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of an siRNA in mice reduces tumor volume (Pille, J., et al (2005) Mol. Ther. 11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55).

For administering an RNAi molecule systemically for the treatment of a disease, the RNAi molecule can be either be modified or alternatively delivered using a drug delivery system; both methods act to prevent the rapid degradation of the RNAi molecule by endo- and exo-nucleases in vivo. Modification of the RNAi molecule or the pharmaceutical carrier can also permit targeting of the RNAi molecule to the target tissue and avoid undesirable off-target effects.

RNA interference molecules can be modified by chemical conjugation to lipophilic groups such as cholesterol to enhance cellular uptake and prevent degradation. For example, an siRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an RNAi molecule to an aptamer has been shown to inhibit tumor growth and mediate tumor regression in a mouse model of prostate cancer (McNamara, J O., et al (2006) Nat. Biotechnol. 24:1005-1015).

In an alternative embodiment, the RNAi molecules can be delivered using drug delivery systems such as e.g., a nanoparticle, a dendrimer, a polymer, liposomes, or a cationic delivery system. Positively charged cationic delivery systems facilitate binding of an RNA interference molecule (negatively charged) and also enhance interactions at the negatively charged cell membrane to permit efficient uptake of an siRNA by the cell. Cationic lipids, dendrimers, or polymers can either be bound to an RNA interference molecule, or induced to form a vesicle or micelle (see e.g., Kim S H., et al (2008) Journal of Controlled Release 129(2):107-116) that encases an RNAi molecule. The formation of vesicles or micelles further prevents degradation of the RNAi molecule when administered systemically. Methods for making and administering cationic-RNAi complexes are well within the abilities of one skilled in the art (see e.g., Sorensen, D R., et al (2003) J. Mol. Biol. 327:761-766; Verma, U N., et al (2003) Clin. Cancer Res. 9:1291-1300; Arnold, A S et al (2007) J. Hypertens. 25:197-205, which are incorporated herein by reference in their entirety).

Some non-limiting examples of drug delivery systems useful for systemic administration of RNAi include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. 25(12):2972-82; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, an RNAi molecule forms a complex with cyclodextrin for systemic administration. Methods for administration and pharmaceutical compositions of RNAi molecules and cyclodextrins can be found in U.S. Pat. No. 7,427,605, which is herein incorporated by reference in its entirety.

The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

One skilled in the art can readily determine the appropriate dose, schedule, and method of administration for the exact formulation of the composition being used, in order to achieve the desired “effective level” in the individual patient. One skilled in the art also can readily determine and use an appropriate indicator of the “effective level” of the compounds of the present invention by a direct (e.g., analytical chemical analysis) or indirect, or analysis of appropriate patient samples (e.g., blood and/or tissues).

Generally, the amount needed is less than the amount needed in antisense treatment applications (see, e.g., Bertrand et al. Biochemical and Biophysical Research Communications 296: 1000-1004, 2002). Antisense therapy has been used in human treatment methods and a skilled artisan may seek additional guidance in dosaging, for example, from publications such as “Results of a Pilot Study Involving the Use of an Antisense Oligodeoxynucleotide Directed Against the Insulin-Like Growth Factor Type I Receptor in Malignant Astrocytomas” by David W. Andrews, et al. in J. Clin Oncol, April 15: 2189-2200, 2001.

The therapeutic compositions of the invention can also be administered to cells ex vivo, e.g., cells are removed from the subject, the compositions comprising the siRNAs or shRNAs of the invention are administered to the cells, and the cells are re-introduced into the subject. Vectors, e.g., gene therapy vectors, can be used to deliver the therapeutic agents to the cells. The cells may be re-introduced into the subject by, for example, intravenous injection.

The prophylactic or therapeutic pharmaceutical compositions of the invention can contain other pharmaceuticals, in conjunction with a vector according to the invention, when used to therapeutically treat obesity, and can also be administered in combination with other pharmaceuticals used to treat obesity.

### Dosage and Administration

In one aspect, the present invention provides a method for treating obesity or preventing triglyceride accumulation in a subject. In one embodiment, the subject can be a mammal. In another embodiment, the mammal can be a human, although the invention is effective with respect to all mammals. The method comprises administering to the subject an effective amount of a pharmaceutical composition comprising a modulator of pyruvate flux distribution, or portion thereof, in a pharmaceutically acceptable carrier. Alternatively, a pharmaceutical composition comprising a nucleic acid inhibitor that shifts pyruvate flux distribution can be administered.

In an important embodiment of the invention, subjects with a BMI greater than 30 are administered an effective amount of a pharmaceutical composition comprising an agent that shifts pyruvate flux distribution in a pharmaceutically acceptable carrier.

In one embodiment, the protein or fragment thereof is linked to a carrier to enhance its bioavailability. Such carriers are known in the art and include poly (alkyl) glycol such as poly ethylene glycol (PEG). Fusion to serum albumin can also increase the serum half-life of therapeutic polypeptides.

Similarly, techniques for making small polypeptides that exhibit activity of larger proteins from which they are derived (in primary sequence) are well known and have become routine in the art. Thus, peptide analogs of proteins that exhibit selective shifts in pyruvate flux distribution, are also useful in the invention.

The dosage ranges for the agent depends upon the form (e.g., protein, small molecule or siRNA), and its potency, and are amounts large enough to produce the desired effect e.g., a reduction in triglyceride accumulation in an adipocyte. The dosage should not be so large as to cause adverse side effects. Generally, the dosage will vary with the age, condition, and sex of the patient and can be determined by one of skill in the art. The dosage can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. Alternatively, the dose range will be titrated to maintain serum levels between 5 μg/mL and 30 μg/mL. An agent can be given once a day, less than once a day multiple times a day, or continuously in order to achieve a therapeutically effective dose.

Administration of the doses recited above can be repeated for a limited period of time. In some embodiments, the doses are given once a day, or multiple doses a day, for example but not limited to three times a day. In a preferred embodiment, the doses recited above are administered daily for several weeks or months. The duration of treatment depends upon the subject's clinical progress and responsiveness to therapy.

A therapeutically effective amount is an amount of an agent that is sufficient to produce a statistically significant, measurable change in triglyceride accumulation (see “Efficacy Measurement” below). Such effective amounts can be gauged in clinical trials as well as animal studies, or isolated adipocyte preparations. A therapeutically effective amount can be assessed in an individual by measuring BMI, weight loss, or decrease in percent fat. Therefore, a therapeutically effective amount also refers to the amount of an agent that is sufficient to cause e.g., a reduction in BMI of at least 0.5 units, or a reduction in percent fat of at least 1%, or a weight loss of at least 5 pounds.

An agent can be administered intravenously by injection or by gradual infusion over time. In general, oral administration is preferred, however any form of administration can be used for the methods described herein. Agents useful in the invention can be administered intravenously, intranasally, orally, by inhalation, transmucosally, intraperitoneally, intramuscularly, subcutaneously, intracavity, transdermally, and can be delivered by peristaltic means, if desired, or by other means known by those skilled in the art.

Therapeutic compositions containing at least one agent can be conventionally administered orally, intravenously, or by injection of a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

An agent may be adapted for catheter-based delivery systems including coated balloons, slow-release drug-eluting stents or other drug-eluting formats, microencapsulated PEG liposomes, or nanobeads for delivery using direct mechanical intervention with or without adjunctive techniques such as ultrasound.

In some embodiments, an agent may be combined with a therapeutically effective amount of another therapeutic agent for treatment of obesity or obesity-related disorders (e.g., metformin to reduce insulin resistance in metabolic syndrome patients).

### Pharmaceutical Compositions

The present invention involves therapeutic compositions useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to a protein or polypeptide with which it is admixed, unless so desired.

The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

For topical application, the carrier may in the form of, for example, and not by way of limitation, an ointment, cream, gel, paste, foam, aerosol, suppository, pad or gelled stick.

### Efficacy Measurement

The efficacy of treatment or the “effective amount” are used interchangeably throughout the specification and can be determined by the skilled clinician using routine methods. However, a treatment is considered “effective treatment,” as the term is used herein, if any one or all of the signs or symptoms of obesity are altered in a beneficial manner, other clinically accepted symptoms are improved, or even ameliorated, e.g., by at least 10% following treatment with at least one agent. Efficacy can also be measured by a failure of an obese individual to develop an obesity-related disease or get worse (i.e., progression of obesity is halted). Methods of measuring these indicators are known to those of skill in the art and/or described herein.

Treatment includes any treatment of a disease in an individual or an animal (some non-limiting examples include a human, or a mammal) and includes: (1) preventing the disease from occurring in an individual which may be predisposed to the disease but does not yet experience or display symptoms of the disease; e.g., prevention of obesity in an individual having lost at least one BMI point; (2) inhibiting the disease, e.g., arresting its development; or (3) relieving the disease, e.g., causing regression of the symptoms or increasing weight loss. An effective amount for the treatment of a disease means that amount which, when administered to a mammal in need thereof, is sufficient to result in effective treatment as that term is defined herein, for that disease.

Efficacy of an agent can be determined by assessing physical indicators of obesity, such as e.g., BMI, waist-to-hip ratio, body fat percentage, total weight loss, percent weight loss, systolic blood pressure, diastolic blood pressure, and waist circumference. In general, a physical parameter (or set of parameters) will be measured in an individual prior to the onset of treatment. The same physical parameter or set thereof, is measured at various time points and compared to the original value of the measured parameter for that individual. A reduction in BMI, waist-to-hip ratio, body fat percentage, total weight or waist circumference are indicators that an agent is efficacious at the current dose regime.

Efficacy of an agent can also be determined by assessing metabolic effects associated with obesity, including e.g., fasting blood glucose, serum triglycerides, insulin, high-density lipoprotein cholesterol, or low-density lipoprotein cholesterol. Metabolic effects can be measured by obtaining e.g., a blood sample from an individual being treated for obesity. A reduction in fasting blood glucose, total serum triglycerides, insulin, or low-density lipoprotein cholesterol indicates that an agent is efficacious at the current dose regime. Conversely, an increase in the level of high-density lipoprotein cholesterol indicates a normalization of metabolic effects due to obesity.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified in the specification and examples are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The present invention may be as defined in any one of the following numbered paragraphs.
1. A method of treating obesity, the method comprising: administering at least one agent that perturbs flux distribution of pyruvate to an obese individual with a BMI greater than 30 in an amount that results in a decrease in triglyceride storage in an adipocyte in the obese individual.
2. The method of paragraph 1, wherein the at least one agent shifts the flux of pyruvate from anaerobic glycolysis to the citric acid cycle.
3. The method of paragraph 1, wherein the at least one agent is selected from the group consisting of: an RNA interference molecule, a small molecule, a protein, a polypeptide, an antibody, an antibody fragment or a metabolite analog.
4. The method of paragraph 1, wherein the at least one agent inhibits at least one enzyme or enzyme complex selected from the group consisting of: lactate dehydrogenase, pyruvate dehydrogenase, pyruvate carboxylase, or any combination thereof.
5. The method of paragraph 3, wherein the at least one agent comprises an inhibitor of pyruvate carboxylase.
6. The method of paragraph 3, wherein the small molecule is oxamate, or phenylacetate.
7. The method of paragraph 3, wherein the RNA interference molecule comprises siRNA.
8. The method of paragraph 1, wherein at least two agents are administered to the obese individual.
9. A method of modifying triglyceride storage in an adipocyte, the method comprising contacting an adipocyte with at least one agent that perturbs flux of pyruvate, wherein the agent decreases storage of triglyceride in the adipocyte.
10. The method of paragraph 9, wherein the at least one agent shifts the flux of pyruvate from anaerobic glycolysis to the citric acid cycle.
11. The method of paragraph 9, wherein the at least one agent is selected from the group consisting of: an RNA interference molecule, a small molecule, a protein, a polypeptide, an antibody, an antibody fragment or a metabolite analog.
12. The method of paragraph 10, wherein the at least one agent inhibits at least one enzyme or enzyme complex selected from the group consisting of: lactate dehydrogenase, pyruvate dehydrogenase, pyruvate carboxylase, or any combination thereof.
13. The method of paragraph 12, wherein the at least one agent comprises an inhibitor of pyruvate carboxylase.
14. The method of paragraph 11, wherein the small molecule is oxamate or phenylacetate.
15. The method of paragraph 11, wherein the RNA interference molecule comprises siRNA.
16. The method of paragraph 9, wherein the adipocyte is contacted with at least two agents that perturb pyruvate flux.
17. A method of modifying triglyceride storage in an adipocyte, the method comprising contacting an adipocyte with at least one inhibitor of pyruvate carboxylase, wherein the inhibitor of pyruvate carboxylase decreases storage of triglyceride in an adipocyte.
18. The method of paragraph 17, wherein the inhibitor shifts the flux of pyruvate from anaerobic glycolysis to the citric acid cycle.
19. The method of paragraph 17, wherein the inhibitor is selected from the group consisting of: an RNA interference molecule, a small molecule, a protein, a polypeptide, an antibody, an antibody fragment or a metabolite analog.
20. The method of paragraph 19, wherein the small molecule is phenylacetate.
21. The method of paragraph 19, wherein the RNA interference molecule comprises siRNA.
22. A method of reducing the number of adipocytes or preventing increase in the number of adipocytes in an individual, the method comprising administering at least one agent that perturbs flux distribution of pyruvate to an individual undergoing a weight loss diet or to an individual who has reduced their body mass index by at least one point, wherein the at least one agent permits a reduction of adipocyte number or prevents an increase in the number of adipocytes in the individual.
23. A method for screening an agent that decreases triglyceride storage in a cell, the method comprising:
   (a) contacting a cell with an agent that perturbs flux of pyruvate, and
   (b) assessing triglyceride levels in the cell,
   wherein if the triglyceride levels in the cell are lower than triglyceride levels in an untreated cell, then the agent decreases storage of triglyceride in the adipocyte.
24. The method of paragraph 23, wherein Oil Red O stain is used to assess triglyceride levels.
25. The method of paragraph 23, wherein the cell comprises an adipocyte or an adipocyte cell line.
26. Use of an agent that perturbs flux of pyruvate for treatment of obesity.
27. The use of paragraph 26, wherein one agent shifts the flux of pyruvate from anaerobic glycolysis to the citric acid cycle.
28. The use of paragraph 26, wherein the agent is selected from the group consisting of: an RNA interference molecule, a small molecule, a protein, a polypeptide, an antibody, an antibody fragment or a metabolite analog.
29. The use of paragraph 26, wherein the agent inhibits at least one enzyme or enzyme complex selected from the group consisting of: lactate dehydrogenase, pyruvate dehydrogenase, pyruvate carboxylase, or any combination thereof.
30. The use of paragraph 26, wherein the agent comprises an inhibitor of pyruvate carboxylase.
31. The use of paragraph 28, wherein the small molecule is oxamate, or phenylacetate.
32. The use of paragraph 28, wherein the RNA interference molecule comprises siRNA.
33. The use of paragraph 28, wherein the agent is used in combination with at least one additional agent.
34. Use of an agent that perturbs flux of pyruvate in the preparation of a medicament to treat obesity.
35. The use of paragraph 34, wherein the agent shifts the flux of pyruvate from anaerobic glycolysis to the citric acid cycle.
36. The use of paragraph 34, wherein the agent is selected from the group consisting of: an RNA interference molecule, a small molecule, a protein, a polypeptide, an antibody, an antibody fragment or a metabolite analog.
37. The use of paragraph 34, wherein the agent inhibits at least one enzyme or enzyme complex selected from the group consisting of: lactate dehydrogenase, pyruvate dehydrogenase, pyruvate carboxylase, or any combination thereof.
38. The use of paragraph 34, wherein the agent comprises an inhibitor of pyruvate carboxylase.
39. The use of paragraph 36, wherein the small molecule is oxamate, or phenylacetate.
40. The use of paragraph 36, wherein the RNA interference molecule comprises siRNA.
41. The use of paragraph 36, wherein the agent is used in combination with at least one additional agent.
42. Use of an agent that perturbs flux of pyruvate for reducing triglyceride storage in an adipocyte.
43. The use of paragraph 42, wherein the agent shifts the flux of pyruvate from anaerobic glycolysis to the citric acid cycle.
44. The use of paragraph 42, wherein the agent is selected from the group consisting of: an RNA interference molecule, a small molecule, a protein, a polypeptide, an antibody, an antibody fragment or a metabolite analog.
45. The use of paragraph 42, wherein the agent inhibits at least one enzyme or enzyme complex selected from the group consisting of: lactate dehydrogenase, pyruvate dehydrogenase, pyruvate carboxylase, or any combination thereof.
46. The use of paragraph 42, wherein the agent comprises an inhibitor of pyruvate carboxylase.
47. The use of paragraph 42, wherein the small molecule is oxamate, or phenylacetate.
48. The use of paragraph 44, wherein the RNA interference molecule comprises siRNA.
49. The use of paragraph 44, wherein the agent is used in combination with at least one additional agent.

### EXAMPLES

It has been previously reported that a redistribution of pyruvate flux from lactate fermentation into the TCA cycle significantly correlated with triglyceride (TG) accumulation in 3T3-L1 adipocytes. The goal of this study was to test the hypothesis that triglyceride accumulation could be altered by specifically perturbing pyruvate metabolism. To address this hypothesis, we treated cultured 3T3-L1 adipocytes with chemical inhibitors of lactate dehydrogenase (LDH) and pyruvate carboxylase (PC), and characterized their global effects on intermediary metabolism using metabolic flux analysis (MFA). Long-term inhibition of lactate dehydrogenase or pyruvate carboxylase (over several days) did not alter the adipocyte differentiation program as assessed by the expression levels of peroxisome proliferator-activated receptor-γ and glycerol-3-phosphate dehydrogenase. Inhibiting lactate dehydrogenase or pyruvate carboxylase up-regulated lipolysis and decreased triglyceride accumulation. Inhibiting PC also up-regulated glycolysis. Metabolic flux analysis indicated that the reduction in triglyceride accumulation was largely explained by decreased de novo fatty acid synthesis, which in turn correlated with lowered anaplerosis. In conclusion, the results of this study indicate that pyruvate acts as a controlling metabolite of triglyceride synthesis. The approach presented herein can help formulate a new strategy to develop therapeutic agents for limiting excessive fat accumulation at the cellular level.

### Example 1

### Methods and Procedures

### Materials

Tissue culture reagents including Dulbecco's Modified Eagle's Medium (DMEM), calf serum (CS), fetal bovine serum (FBS), human insulin, and penicillin/streptomycin were purchased from Invitrogen (Carlsbad, Calif.). Unless otherwise noted, all other chemicals were purchased from SIGMA™ (St. Louis, Mo.).

### Cell Culture and Differentiation

3T3-L1 preadipocytes were plated onto 24-well plates and expanded in preadipocyte growth medium consisting of DMEM supplemented with calf serum (10% v/v), penicillin and streptomycin. Medium was replenished every other day. Two days post-confluence, the cells were induced to differentiate using an adipogenic cocktail (1 μg/ml insulin, 0.5 mM isobutylmethylxanthine, and 1 μM dexamethasone) added to a basal medium (DMEM with 10% fetal bovine serum and penicillin/streptomycin). After 48 hrs, the first medium was replaced with a second induction medium consisting of the basal adipocyte medium supplemented with only insulin. After another 48 hrs, the second medium was replaced with the adipocyte basal medium containing different concentrations of oxamate or phenylacetate. This basal medium was replenished every other day during the remainder of the culture experiments.

### Microscopy

At the indicated time points, cellular morphology was recorded using phase-contrast microscopy (Nikon-US, Melville, N.Y.). The images were analyzed using the SimplePCI software package (Compix Inc., Cranberry Township, Pa.). Intracellular lipid droplets were visualized by staining with Oil Red O as described previously (Hauner H, Skurk T, Wabitsch M. 2001. In: Adipose tissue protocols. Ailhaud G, ed.; Humana Press: Totowa, N.J.: xiii, 334).

### Real-Time RT PCR

Total RNA was isolated using the RNEASY MINI KIT® RNA isolation kit from QIAGEN® (Valencia, Calif.). Reverse transcription was performed on a PTC-100 Programmable Thermal Controller (MJ Research, Waltham, Mass.) using the High-Capacity cDNA Archive Kit (Applied Biosystems, Foster City, Calif.) with random primers. The peroxisome proliferator-activated receptor-γ (PPAR-γ) and glycerol-3-phosphate dehydrogenase (EC 1.1.1.8, GPDH) mRNA and 18S rRNA levels were determined using the TAQMAN® Gene Expression assay (Applied Biosystems, Foster City, Calif.). All gene expression data were normalized to the 18S rRNA contents in corresponding samples. Fold-changes with respect to untreated control were calculated using the normalized data.

### Metabolite Assays

Metabolite measurements were performed both on cell lysates and spent medium samples as described previously (Si Y, Yoon J, Lee K. American journal of physiology 2007; 292:E1637-46). Cells were lysed in situ with an 0.1% SDS buffer and sonicated. Immediately after collection, the spent medium samples were cleaned of cell debris by a brief centrifugation step. Free glycerol, TG and free fatty acids (FFAs) were measured using enzymatic assay kits (SIGMA™). Glucose and lactate concentrations were measured using the methods of Trinder (Trinder P. J Clin Pathol 1969; 22:158-61) and Loomis (Loomis M E. J Lab Clin Med 1961; 57:966-9), respectively. Amino acids were quantified by HPLC using fluorescence-based detection following pre-column derivatization of primary or secondary amines with 6-aminoquinolyl-N-hydroxysuccinimidyl-carbamate (Cohen S A, De Antonis K M. J Chromatogr A 1994; 661:25-34). All metabolite data were normalized by the corresponding cell sample DNA content, which was determined with a fluorescence-based assay using the Hoechst dye.

### Oxygen Uptake

The dissolved oxygen in the culture medium was measured using a needle-type fiber-optic microsensor (MicroxTX3, Presens, Regensburg, Germany). The concentration data were related to the oxygen uptake rate (OUR) using the following diffusion-reaction model:

$$\frac{{d}^{2} c}{d {z}^{2}} = 0 , B . C . 1 \text{:} c ( z = 0 ) = {c}_{0} , B . C . 2 \text{:} - {D}_{{o}_{2}} \frac{d c}{d {z}^{2}} {\vert}_{z = - {h}_{b}} = {J}_{{o}_{2}} \qquad ( 1 )$$

where c₀ and h_{b} are, respectively, the oxygen concentration at medium surface and height of the medium in the culture well. The bottom of the culture plate (and cell monolayer) is at z=−h_{b}. Integrating equation (1) and applying the boundary conditions we obtain:

$${J}_{{o}_{2}} = \frac{{D}_{{o}_{2}}}{{h}_{m}} ( {c}_{0} - {c}_{m} ) \qquad ( 2 )$$

where hₘ and cₘ are, respectively, the distance between the probe and the medium surface and the measured oxygen concentration in the medium at the probe depth. The oxygen uptake rate was calculated by dividing the flux J_{O2} by the cell density. The measured oxygen uptake rate was used for relative comparisons between the treatment groups and not used for MFA in order to independently confirm the calculated oxygen uptake rate.

### Stoichiometric Model and Flux Analysis

A stoichiometric model of adipocyte central carbon metabolism was constructed as described previously (Si Y, Yoon J, Lee K. American journal of physiology 2007; 292:E1637-46). The model consisted of the major pathways of central carbon metabolism (Table 1). Intracellular fluxes were estimated by solving a constrained non-linear optimization problem with stoichiometric and thermodynamic constraints (Nolan R P, Fenley A P, Lee K. Metab Eng 2006; 8:30-45).

TABLE 1
Reaction stoichiometry of the model adipocyte network

| RXN | Pathway | Stoichiometry |
|---|---|---|
| 1 | Glycolysis | Glucose + ATP = Glucose 6-P + ADP |
| 2 | Glycolysis | Glucose 6-P = Fructose 6-P |
| 3 | Glycolysis | Fructose 6-P + ATP = Glyceraldehyde 3-P + Glycerone-P + ADP |
| 4 | Glycolysis | Glycerone-P = Glyceraldehyde 3-P |
| 5 | Glycolysis | Glyceraldehyde 3-P + NAD+ + ADP + Pi = P-Enolpyruvate + |
| | | ATP + H2O |
| 6 | Glycolysis | P-Enolpyruvate + ADP + H+ = Pyruvate + ATP |
| 7 | Glycolysis | Pyruvate + NADH + H+ = Lactate + NAD+ |
| 8 | Pentose phosphate pathway | Glucose 6-P + 2 NADP+ + H2O = Ribulose 5-P + CO2 + 2 |
| | | NADPH + 2 H+ |
| 9 | Pentose phosphate pathway | 3 Ribulose 5-P = 2 Fructose 6-P + Glyceraldehyde 3-P |
| 10 | TCA cycle (mitochondria) | [symbol] + [symbol] + NAD+ + H2O = [symbol] + CO2 + |
| | | NADH + H+ |
| 11 | TCA cycle (mitochondria) | [symbol] + HCO3− + ATP = [symbol] + ADP + Pi |
| 12 | TCA cycle (mitochondria) | [symbol] + NAD+ = [symbol] + CO2 + NADH + H+ |
| 13 | TCA cycle (mitochondria) | [symbol] + NAD+ + CoA = [symbol] + CO2 + |
| | | NADH |
| 14 | TCA cycle (mitochondria) | [symbol] + FAD + Pi + ADP = [symbol] + FADH2 + ATP + |
| | | CoA |
| 15 | TCA cycle (mitochondria) | [symbol] + H2O = [symbol] |
| 16 | TCA cycle (mitochondria) | [symbol] + NAD+ = [symbol] + NADH + H+ |
| 17 | TCA cycle | Citrate + CoA + ATP = Acetyl-CoA + Oxaloacetate + ADP + Pi |
| 18 | TCA cycle | Oxaloacetate + NADH + H+ = Malate + NAD+ |
| 19 | TCA cycle | Malate + NADP+ = Pyruvate + CO2 + NADPH |
| 20 | TCA cycle | Citrate + NADP+ = 2-Oxoglutarate + CO2 + NADPH + H+ |
| 21 | TCA cycle | Oxaloacetate + ATP = P-Enolpyruvate + CO2 + ADP |
| 22 | Oxidative phosphorylation | NADH + 0.5 O2 + 3 ADP + 3 Pi + 4 H+ = NAD+ + 3 ATP + 4 |
| | | H2O |
| 23 | Oxidative phosphorylation | FADH2 + 0.5 O2 + 2 ADP + 2 Pi + 3 H+ = FAD + 2 ATP + 3 |
| | | H2O |
| 24 | Palmitate biosynthesis | 8 Acetyl-CoA + 14 NADPH + 7 ATP + 7 HCO3− + 14 H+ = |
| | | Palmitate + 14 NADP+ + 8 CoA + 7 ADP + 7 Pi + 7 CO2 + 6 |
| | | H2O |
| 25 | Tripalmitoylglycerol | Glycerone-P + 3 Palmitate + NADH + 3 ATP + H2O + H+ = |
| | biosynthesis | Tripalmitoylglycerol + NAD+ + Pi + 3 AMP + 3 PPi |
| 26 | Tripalmitoylglycerol | Tripalmitoylglycerol + 3 H2O = Glycerol + 3 Palmitate |
| | biosynthesis | |
| 27 | Tripalmitoylglycerol | Tripalmitoylglycerol = Tripalmitoylglycerol |
| | accumulation | |
| 28 | Metabolism of ketone | 2 [symbol] = [symbol] + 2 CoA |
| | bodies | |
| 29 | Metabolism of ketone | [symbol] = [symbol] + CoA |
| | bodies | |
| 30 | Metabolism of ketone | [symbol] + NADH = 3-Hydroxybutyrate |
| | bodies | |
| 31 | Amino acid metabolism | Pyruvate + NH4+ + NADPH = Alanine |
| 32 | Amino acid metabolism | Aspartate + NH4+ = Asparagine |
| 33 | Amino acid metabolism | Aspartate = Oxaloacetate + NH4+ + NADH |
| 34 | Amino acid metabolism | Cysteine = Pyruvate + NH4+ + NADH |
| 35 | Amino acid metabolism | Glutamate = 2-Oxoglutarate + NH4+ + NADH |
| 36 | Amino acid metabolism | Glutamate + NH4+ + ATP = Glutamine |
| 37 | Amino acid metabolism | Serine + THF = Glycine |
| 38 | Amino acid metabolism | Histidine + THF = Glutamate + NH4+ |
| 39 | Amino acid metabolism | Isoleucine + 2 CoA = [symbol] + [symbol] + NH4+ + |
| | | FADH2 + 2 NADH |
| 40 | Amino acid metabolism | Leucine + CoA + CO2 + ATP = [symbol] + [symbol] + |
| | | NH4+ + FADH2 + 2 NADH |
| 41 | Amino acid metabolism | Lysine = 2-Oxoadipate + 2 NH4+ + 3 NADH |
| 42 | Amino acid metabolism | 2-Oxoadipate + CoA = [symbol] + 2 CO2 + FADH2 + 2 |
| | | NADH |
| 43 | Amino acid metabolism | Methionine + Serine + ATP + CoA + THF = [symbol] + |
| | | Cysteine + NH4+ + NADH |
| 44 | Amino acid metabolism | Phenylalanine + O2 + NADH = Tyrosine |
| 45 | Amino acid metabolism | Glutamate + ATP + 2 NADPH = Proline |
| 46 | Amino acid metabolism | Serine = Pyruvate + NH4+ |
| 47 | Amino acid metabolism | Threonine + CoA = Glycine + [symbol] + NADH |
| 48 | Amino acid metabolism | Tryptophan + 3 O2 + NADPH = 2-Oxoadipate + Alanine + CO2 + |
| | | NH4+ |
| 49 | Amino acid metabolism | Tyrosine + 2 O2 = [symbol] + [symbol] + CO2 + NH4+ + |
| | | NADH |
| 50 | Amino acid metabolism | Valine + CoA = [symbol] + CO2 + 4 NADH + FADH2 + |
| | | NH4+ |
| 51 | Plasma exchange | Palmitate = Palmitate |
| 52 | Plasma exchange | [symbol] = Acetoacetate |
| 53 | Plasma exchange | Alanine = Alanine |
| 54 | Plasma exchange | Aspartate = Aspartate |
| 55 | Plasma exchange | Cysteine = Cysteine |
| 56 | Plasma exchange | Glutamate = Glutamate |
| 57 | Plasma exchange | Glycine = Glycine |
| 58 | Plasma exchange | Serine = Serine |
| 59 | Plasma exchange | Tyrosine = Tyrosine |
| 60 | Plasma exchange | O2 = O2 |
| 61 | Plasma exchange | CO2 = CO2 |
| 62 | Plasma exchange | NH4+ = NH4+ |
| 63 | Mitochondrial exchange | Pyruvate = [symbol] |
| 64 | Mitochondrial exchange | [symbol] + Malate = Citrate + [symbol] |
| 65 | Mitochondrial exchange | 2-Oxoglutarate + [symbol] = [symbol] + Malate |
| 66 | Mitochondrial exchange | [symbol] + Pi = Malate + [symbol] |

Extracellular metabolites are indicated in bold. Mitochondrial metabolites are indicated in bold italics. Several entries in the table (e.g. reaction no. 10) represent pseudo-reactions obtained by condensing sequences of non-branching reactions.

### Statistics

Comparisons between two experimental groups were performed using one-way ANOVA. Group means were deemed to be statistically significantly different when p<0.05.

### Example 2

### Impact of Perturbed Pyruvate Metabolism on Adipocyte Triglyceride Accumulation

### Proliferation and Differentiation of Adipocytes

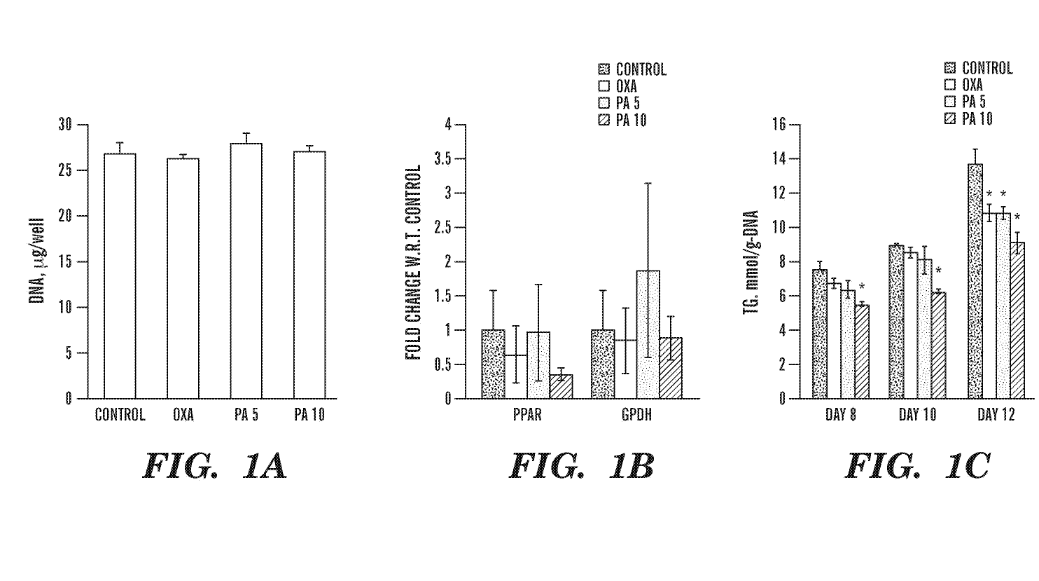

Chemical inhibitors were first added to the culture medium on day 4 post-induction to avoid interfering with the early differentiation program of the 3T3-L1 cells. Treatment with the chemical inhibitors at the indicated levels did not significantly affect cell number (FIG. 1A). Differentiation was assessed by measuring the mRNA expression of PPAR-γ and GPDH on day 8 post-induction (Brun R P, Tontonoz P, Forman B M, et al. Genes Dev 1996; 10:974-84; Pairault J, Green H. Proc Natl Acad Sci USA 1979; 76:5138-42). The inhibitors had no statistically significant effect on the expression of either gene (FIG. 1B).

### Reduction in TG Accumulation in Adipocytes

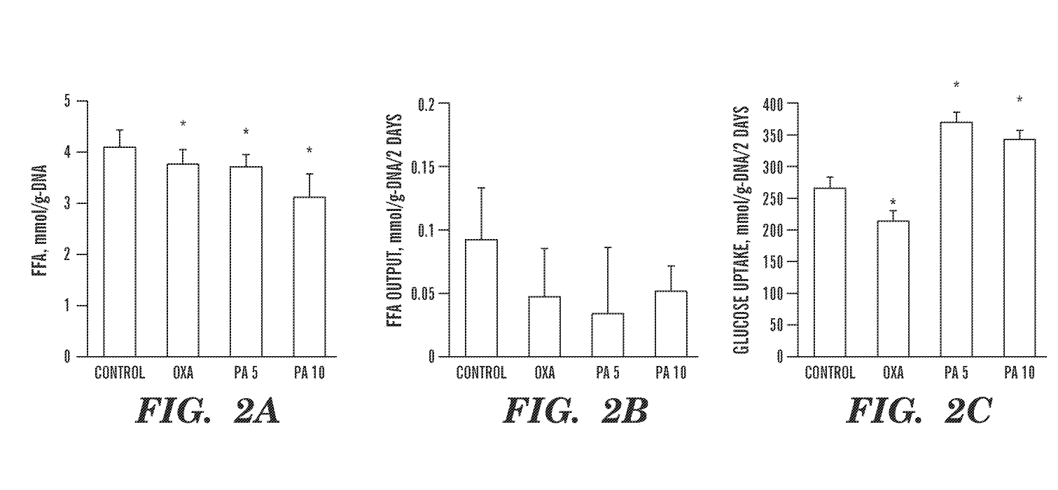

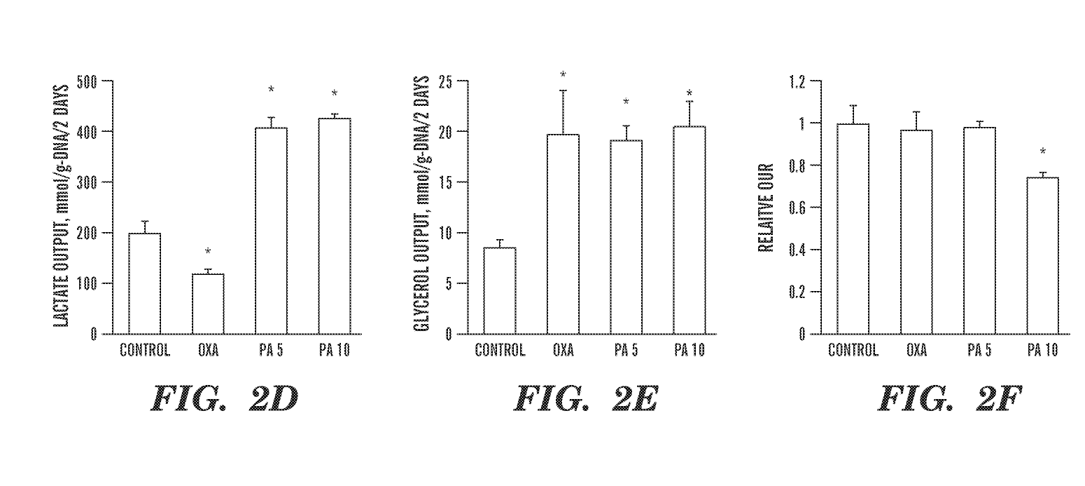

The amount of intracellular triglyceride in the adipocytes was monitored at different time points after beginning the inhibitor treatments (FIG. 1C). The largest differences relative to the untreated control were observed on day 12 post-induction, when the adipocytes were expected to have achieved their mature phenotype. Based on this observation, the remainder of the study focused on metabolite data collected between days 10 and 12 post-induction. Morphological assessment showed that the induced cells were indeed round and contained visible lipid droplets (Green H, Kehinde O. Cell 1975; 5:19-27). Lipid levels were assessed in adipocytes by Oil-Red O staining of 3T3-L1 cells on day 12 post-induction. Oil red O staining was performed in control treated adipocytes, adipocytes treated with 20 mM oxamate, adipocytes treated with 5 mM phenylacetate, and adipocytes treated with 10 mM phenylacetate. Oil Red O staining visually confirmed that adipocytes treated with oxamate, and phenylacetate contained less lipid than the untreated controls. The reduction in triglyceride on day 12 varied from 21˜34% depending on the inhibitor type and concentration, with 10 mM phenylacetate having the largest effect (FIG. 1C). The intracellular free fatty acid concentration (FIG. 2A) was unaffected by oxamate, but significantly reduced by 9 and 17% with 5 and 10 mM phenylacetate treatment, respectively.

### Metabolite Profile

To examine whether the chemical inhibitors also affected other parts of adipocyte metabolism, the uptake or output of other primary metabolites on day 12 post-induction was measured. The focus was on the profiles of glucose, lactate, glycerol, free fatty acids and oxygen. The amino acids (Table 2) were utilized to smaller extents, accounting for less than 10% of total carbon uptake. The inhibitory effect of oxamate on lactate dehydrogenase was dose-dependent (data not shown). At 20 mM, oxamate significantly inhibited lactate output by 40% compared to untreated control (FIG. 2D). This treatment also significantly reduced glucose uptake by 20% (FIG. 2C). The largest effect was observed for glycerol output, which was raised 1.3-fold (FIG. 2E). Oxygen consumption was not significantly affected by oxamate treatment (FIG. 2F). Treatment with 5 and 10 mM phenylacetate significantly increased glucose uptake by 38 and 28%, respectively (FIG. 2C) and lactate output by more than 2-fold (FIG. 2D). Treatment with 5 and 10 mM phenylacetate also significantly increased glycerol output by 1.2- and 1.4-fold, respectively (FIG. 2E). Oxygen consumption was significantly affected (25% reduction) at the higher concentration (FIG. 2F). The net release of free fatty acids into the medium was negligible and not significantly different across all treatment conditions (FIG. 2B).

TABLE 2
Amino acid uptake or output on day 12 post-induction.

| Amino | | | | |
|---|---|---|---|---|
| Acid | Control | 20 mM OXA | 5 mM PA | 10 mM PA |
| ALA | 0.7 ± 0.0 | 0.6 ± 0.0 * | 0.4 ± 0.1 * | −0.5 ± 0.1 * |
| ASN | −3.0 ± 0.1 | −1.1 ± 0.2 * | −4.6 ± 0.2 * | −5.9 ± 0.4 * |
| ASP | 0.0 ± 0.0 | 0.0 ± 0.0 | 0.0 ± 0.0 | 0.0 ± 0.0 |
| GLN | −11.2 ± 1.4 | −12.0 ± 1.5 | −7.9 ± 0.6 * | 2.6 ± 2.9 * |
| GLU | 0.4 ± 0.0 | 0.4 ± 0.0 * | 0.3 ± 0.0 * | 0.3 ± 0.0 * |
| GLY | −8.8 ± 0.4 | −5.9 ± 0.4 * | −7.3 ± 0.2 * | −7.9 ± 0.9 |
| HIS | −0.4 ± 0.1 | −0.2 ± 0.1 * | −0.1 ± 0.1 * | −0.1 ± 0.2 * |
| ILE | 8.2 ± 0.2 | 6.1 ± 0.3 * | 8.1 ± 0.3 | 7.5 ± 0.2 * |
| LEU | 8.8 ± 0.2 | 6.8 ± 0.3 * | 8.6 ± 0.3 | 7.9 ± 0.2 * |
| LYS | 0.3 ± 0.5 | −2.0 ± 0.3 * | −1.8 ± 0.1 * | −2.0 ± 0.2 * |
| MET | 0.1 ± 0.1 | 0.0 ± 0.0 | 0.1 ± 0.1 | 0.1 ± 0.2 |
| PHE | −0.8 ± 0.1 | −0.3 ± 0.1 * | −0.1 ± 0.1 * | −0.1 ± 0.4 * |
| PRO | −0.4 ± 0.1 | −0.5 ± 0.0 | −1.4 ± 0.0 * | −2.1 ± 0.2 * |
| SER | 4.1 ± 0.1 | 4.0 ± 0.2 | 4.5 ± 0.2 * | 4.6 ± 0.5 |
| THR | −1.0 ± 0.3 | −1.4 ± 0.3 | −1.2 ± 0.1 | −1.4 ± 0.4 |
| TYR | −0.7 ± 0.1 | −0.3 ± 0.1 * | 0.1 ± 0.2 * | −0.0 ± 0.4 * |
| VAL | 5.5 ± 0.2 | 3.3 ± 0.2 * | 5.1 ± 0.4 | 3.8 ± 0.4 * |

All units are in mmol/g-DNA/2 days. Data shown are means ± SD (n = 6).
* statistically significantly different from control (p < 0.05).
Positive value indicates uptake.

### Metabolic Flux Analysis

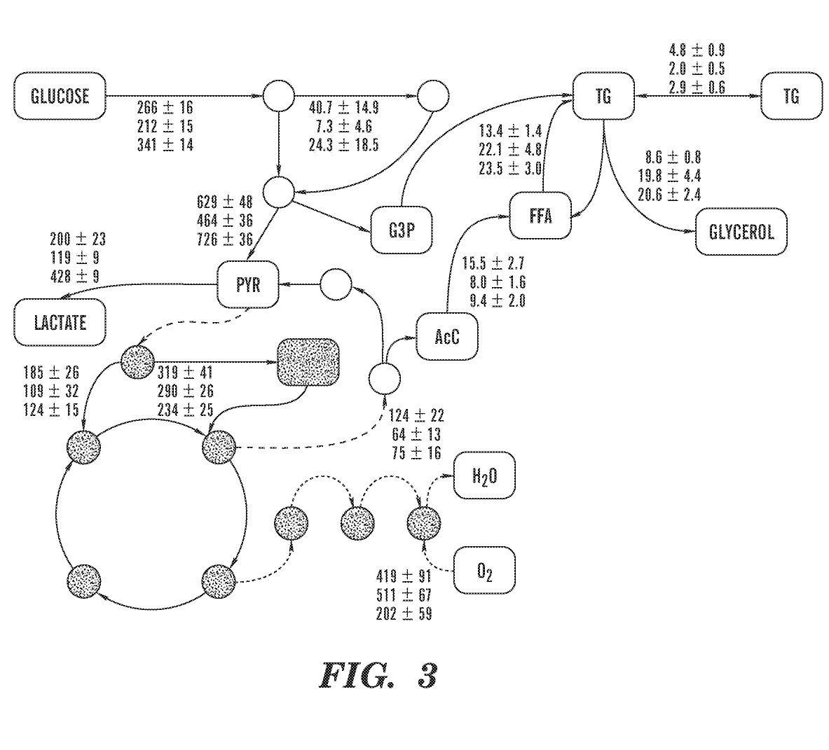

Metabolic flux analysis was applied to integrate the metabolite measurements and quantify the broader impact of the inhibitors on adipocyte intermediary metabolism. FIG. 3 summarizes the estimated fluxes through the major pathways. The complete flux data are shown in Table 3. As suggested by the glucose profile, oxamate down-regulated the flux through glycolysis (17˜26%). In addition, flux through the pentose phosphate pathway (PPP) (82%), pyruvate flux entering the TCA cycle (21%), PC flux (41%) and citrate transport out of the mitochondria (44%) were all decreased. The reactions of the TCA cycle and oxidative phosphorylation were not significantly affected. While de novo fatty acid synthesis decreased by 49%, triglyceride synthesis and lipolysis both increased by 65 and 131%, respectively, reducing net triglyceride accumulation by 52%. Estimates of oxygen uptake ratio obtained through the flux model showed no statistical difference between control and oxamate treatment, in good agreement with the measurements.

Treatment with phenylacetate raised the flux through glycolysis by 15˜60%, but lowered the flux through the PPP by 83%. The lowered PPP flux was noted only for the 5 mM condition. The total flux of pyruvate into the mitochondrial reactions was not affected at 5 mM, but significantly decreased (29%) at the higher concentration. The pyruvate carboxylase flux decreased by 23 and 33% in the 5 and 10 mM phenylacetate conditions, respectively. At 10 mM phenylacetate, PDC flux also decreased by 27%. Citrate transport out of the mitochondria also decreased, by 40 and 46% in the 5 and 10 mM phenylacetate conditions, respectively. The other reactions of the TCA cycle were not affected. The calculated oxygen uptake ratio was in qualitative agreement with the experimental results. A significant difference compared to control was estimated and measured only for the 10 mM condition, where the measured oxygen uptake ratio of the treated cells was lowered by 52%. In the 10 mM condition, the calculated CO₂ output was also lower by 23%. Finally, the phenylacetate treatment reduced de novo fatty acid synthesis by ˜40%. The fluxes through other lipogenic reactions (including esterification) were increased 64˜75%, with the higher concentration having the larger effect. The effect on lipolysis was larger, with increases of 1.2- and 1.4-fold, respectively, in cultures treated with 5 and 10 mM PA. Flux through phosphoenolpyruvate carboxykinase, a key step in glycerogenesis, also decreased by 40%. The net result was that triglyceride accumulation was reduced by 40%.

TABLE 3
Metabolic flux profiles on day 12 post-induction.

| RXN. | Control | OXA 20 mM | PA 5 mM | PA 10 mM |
|---|---|---|---|---|
| 1 | 266.0 ± 16.2 | 212.3 ± 15.3 * | 367.0 ± 17.6 * | 341.0 ± 14.4 * |
| 2 | 225.3 ± 12.9 | 205.0 ± 14.2 * | 359.9 ± 23.3 * | 316.7 ± 19.4 * |
| 3 | 252.4 ± 13.4 | 209.9 ± 14.8 * | 364.7 ± 19.3 * | 332.9 ± 13.7 * |
| 4 | 239.0 ± 12.8 | 187.8 ± 13.9 * | 342.7 ± 18.4 * | 309.4 ± 13.0 * |
| 5 | 505.1 ± 28.5 | 400.1 ± 28.6 * | 709.8 ± 35.8 * | 650.5 ± 26.0 * |
| 6 | 629.4 ± 47.6 | 463.5 ± 35.6 * | 784.6 ± 35.5 * | 725.6 ± 35.8 * |
| 7 | 200.0 ± 23.5 | 119.2 ± 9.1 * | 410.4 ± 19.1 * | 427.8 ± 9.4 * |
| 8 | 40.7 ± 14.9 | 7.3 ± 4.6 * | 7.1 ± 7.8 * | 24.3 ± 18.5 |
| 9 | 13.6 ± 5.0 | 2.4 ± 1.5 * | 2.4 ± 2.6 * | 8.1 ± 6.2 |
| 10 | 318.7 ± 41.3 | 290.4 ± 25.6 | 312.7 ± 48.2 | 233.7 ± 25.1 * |
| 11 | 184.6 ± 26.1 | 109.1 ± 31.7 * | 141.4 ± 13.2 * | 123.5 ± 15.3 * |
| 12 | 132.2 ± 32.6 | 185.0 ± 19.7 * | 200.9 ± 54.1 * | 132.2 ± 19.3 |
| 13 | 194.4 ± 32.7 | 226.7 ± 26.2 | 237.8 ± 52.0 | 158.6 ± 26.1 |
| 14 | 208.1 ± 32.6 | 236.0 ± 26.3 | 251.0 ± 52.6 | 169.9 ± 26.6 |
| 15 | 208.1 ± 32.6 | 236.0 ± 26.3 | 251.2 ± 52.7 | 170.1 ± 27.0 |
| 16 | 134.2 ± 34.7 | 181.2 ± 40.8 | 171.3 ± 45.2 | 110.2 ± 17.8 |
| 17 | 124.4 ± 21.8 | 63.7 ± 12.9 * | 74.9 ± 9.0 * | 75.1 ± 16.4 * |
| 18 | 0.1 ± 0.1 | 0.3 ± 0.8 | 0.0 ± 0.0 | 0.0 ± 0.0 |
| 19 | 74.0 ± 12.1 | 55.2 ± 20.8 | 79.9 ± 9.7 | 59.9 ± 9.2 * |
| 20 | 62.2 ± 20.0 | 41.6 ± 9.1 | 36.9 ± 7.3 * | 26.4 ± 6.9 * |
| 21 | 124.3 ± 21.9 | 63.4 ± 12.9 * | 74.8 ± 9.0 * | 75.1 ± 16.4 * |
| 22 | 607.9 ± 150.0 | 770.4 ± 108.2 | 527.6 ± 214.3 | 213.9 ± 89.2 * |
| 23 | 230.8 ± 32.6 | 252.2 ± 26.4 | 272.8 ± 53.3 | 189.1 ± 27.3 * |
| 24 | 15.5 ± 2.7 | 8.0 ± 1.6 * | 9.4 ± 1.1 * | 9.4 ± 2.0 * |
| 25 | 13.4 ± 1.4 | 22.1 ± 4.8 * | 22.0 ± 1.3 * | 23.5 ± 3.0 * |
| 26 | 8.6 ± 0.8 | 19.8 ± 4.4 * | 19.2 ± 1.4 * | 20.6 ± 2.4 * |
| 27 | 4.8 ± 0.9 | 2.3 ± 0.5 * | 2.8 ± 0.4 * | 2.9 ± 0.6 * |
| 28 | 8.9 ± 0.2 | 6.4 ± 0.3 * | 8.4 ± 0.3 * | 7.8 ± 0.2 * |
| 29 | 0.3 ± 0.4 | 0.0 ± 0.0 | 0.0 ± 0.0 | 0.0 ± 0.0 |
| 30 | 16.5 ± 1.3 | 10.8 ± 2.2 * | 17.0 ± 0.6 | 15.9 ± 0.7 |
| 31 | 0.0 ± 0.0 | 0.0 ± 0.0 | 0.0 ± 0.0 | 0.5 ± 0.1 * |
| 32 | 1.5 ± 0.1 | 0.6 ± 0.1 * | 2.3 ± 0.1 * | 2.9 ± 0.2 * |
| 33 | 0.0 ± 0.0 | 0.0 ± 0.0 | 0.0 ± 0.0 | 0.0 ± 0.0 |
| 34 | 0.0 ± 0.0 | 0.0 ± 0.0 | 0.0 ± 0.0 | 0.0 ± 0.0 |
| 35 | 0.0 ± 0.0 | 0.0 ± 0.0 | 0.0 ± 0.0 | 0.0 ± 0.0 * |
| 36 | 7.5 ± 0.9 | 8.0 ± 1.0 | 5.3 ± 0.4 * | 0.0 ± 0.0 * |
| 37 | 6.0 ± 0.3 | 5.0 ± 0.3 * | 5.8 ± 0.2 | 6.2 ± 0.4 |
| 38 | 3.8 ± 0.5 | 4.1 ± 0.5 | 2.7 ± 0.2 * | 0.5 ± 0.1 * |
| 39 | 8.2 ± 0.2 | 6.1 ± 0.3 * | 8.1 ± 0.3 | 7.5 ± 0.2 * |
| 40 | 8.8 ± 0.2 | 6.8 ± 0.3 * | 8.6 ± 0.3 | 7.9 ± 0.2 * |
| 41 | 0.3 ± 0.4 | 0.0 ± 0.0 | 0.0 ± 0.0 | 0.0 ± 0.0 |
| 42 | 0.3 ± 0.4 | 0.0 ± 0.0 | 0.0 ± 0.0 | 0.0 ± 0.0 |
| 43 | 0.0 ± 0.0 | 0.0 ± 0.0 | 0.0 ± 0.0 | 0.0 ± 0.0 |
| 44 | 0.0 ± 0.0 | 0.0 ± 0.0 | 0.0 ± 0.0 | 0.1 ± 0.3 |
| 45 | 0.0 ± 0.0 | 0.0 ± 0.0 * | 0.0 ± 0.0 | 1.5 ± 0.1 * |
| 46 | 0.0 ± 0.0 | 0.0 ± 0.0 | 0.0 ± 0.0 | 0.0 ± 0.0 |
| 47 | 0.9 ± 0.1 | 0.0 ± 0.0 * | 0.1 ± 0.1 * | 0.2 ± 0.2 * |
| 48 | 0.0 ± 0.0 | 0.0 ± 0.0 | 0.0 ± 0.0 | 0.0 ± 0.0 |
| 49 | 0.0 ± 0.0 | 0.0 ± 0.0 | 0.2 ± 0.1 | 0.2 ± 0.5 |
| 50 | 5.5 ± 0.2 | 3.3 ± 0.2 * | 5.1 ± 0.4 | 3.8 ± 0.4 * |
| 51 | 1.1 ± 0.1 | 1.0 ± 0.1 | 1.0 ± 0.1 * | 0.8 ± 0.1 * |
| 52 | 1.5 ± 1.2 | 2.4 ± 1.9 | 0.2 ± 0.2 * | 0.0 ± 0.1 * |
| 53 | 0.0 ± 0.0 | 0.0 ± 0.0 | 0.0 ± 0.0 | 0.5 ± 0.1 * |
| 54 | 1.5 ± 0.1 | 0.6 ± 0.1 * | 2.3 ± 0.1 * | 2.9 ± 0.2 * |
| 55 | 0.0 ± 0.0 | 0.0 ± 0.0 | 0.0 ± 0.0 | 0.0 ± 0.0 |
| 56 | 3.6 ± 0.4 | 3.9 ± 0.5 | 2.6 ± 0.2 * | 0.9 ± 0.1 * |
| 57 | 6.9 ± 0.2 | 5.0 ± 0.3 * | 5.9 ± 0.2 * | 6.4 ± 0.4 * |
| 58 | 6.0 ± 0.3 | 5.0 ± 0.3 * | 5.8 ± 0.2 | 6.2 ± 0.4 |
| 59 | 0.0 ± 0.0 | 0.0 ± 0.0 | 0.1 ± 0.1 * | 0.1 ± 0.2 |
| 60 | 419.3 ± 91.2 | 511.3 ± 67.1 | 400.5 ± 134.0 | 202.1 ±59.1 * |
| 61 | 759.2 ± 105.9 | 756.9 ± 75.2 | 805.5 ± 145.9 | 582.8 ± 66.5 * |
| 62 | 17.9 ± 1.7 | 11.7 ± 0.5 * | 17.0 ± 1.0 | 16.5 ± 1.3 |
| 63 | 503.3 ± 59.8 | 399.5 ± 40.8 * | 454.0 ± 54.4 | 357.2 ± 37.5 * |
| 64 | 186.6 ± 39.0 | 105.3 ± 13.9 * | 111.7 ± 15.8 * | 101.5 ± 16.0 * |
| 65 | 62.2 ± 20.0 | 41.6 ± 9.1 | 36.9 ± 7.3 * | 26.4 ± 6.9 * |
| 66 | 73.9 ± 12.1 | 54.8 ± 20.5 | 79.9 ± 9.7 | 59.9 ± 9.2 |

All units are in mmol/g-DNA/2 days. Data shown are means ± SD (n = 6).
* statistically significantly different from untreated control (p < 0.05).
Reaction numbers refer to Table 1.

A previous study on the metabolic profile of in vitro 3T3-L1 adipogenesis pointed to a correlation between a shift in the flux distribution around pyruvate and induction of lipogenic activity (Si Y, Yoon J, Lee K. American journal of physiology 2007; 292:E1637-46). Herein, chemical inhibitors were used to investigate whether specifically perturbing pyruvate metabolism affects triglyceride accumulation. The two inhibitors, oxamate and phenylacetate, both inhibited the targeted reactions in a dose-dependent fashion. Although these inhibitors work acutely, the goal was to achieve sustained down-regulation of the target reaction fluxes. Therefore, the inhibitors were administered over a prolonged period from day 4, when visible lipid droplets first appeared, to day 12 post-induction, when the induced 3T3-L1 cells were considered mature adipocytes. Indeed, the relative reduction in triglyceride accumulation correlated with the treatment duration (FIG. 1C). This effect did not appear to reflect an interference of the normal differentiation program, as there were no significant effects on the cell number (FIG. 1A) and gene expression levels of GPDH and PPAR-γ (FIG. 1B).

A previously developed MFA model (Si Y, Yoon J, Lee K. American journal of physiology 2007; 292:E1637-46) was applied to characterize the global effects of the chemical inhibitors on adipocyte intermediary metabolism. To validate the qualitative accuracy of the model, the calculated and experimentally determined oxygen uptake rate was compared across the different treatment groups and found good agreement. Flux calculations (Table 3, reaction #60) correctly predicted that the inhibitors either decreased (10 mM P phenylacetate condition) or did not affect (oxamate and 5 mM phenylacetate conditions) the oxygen uptake rate. Model estimates were also corroborated by reports dealing with the effects of lactate dehydrogenase or PC inhibition on individual pathways. For example, inhibiting lactate dehydrogenase was found to decrease glucose uptake, consistent with prior reports on the effects of oxamate on tumor (HeLa S3) cell glycolysis (Goldberg E B, Colowick S P. J Biol Chem 1965; 240:2786-90; Goldberg E B, Nitowsky H M, Colowick S P. J Biol Chem 1965; 240:2791-6). Similar results were obtained in Chinese hamster ovary cells when lactate dehydrogenase was down-regulated by homologous recombination (Chen K, Liu Q, Xie L, Sharp P A, Wang D I. Biotechnol Bioeng 2001; 72:55-61) or siRNA treatment (Kim S H, Lee G M. Appl Microbiol Biotechnol 2007; 74:152-9). One possible explanation involves the cytosolic redox ratio. Inhibiting lactate dehydrogenase increases the NADH:NAD⁺ ratio, which can inhibit the NAD⁺-dependent conversion of glyceraldehyde 3-phosphate (GAP) into 3-phosphoglycerate, lower glycolytic ATP production, reduce the activities of hexokinase and fructose-6-phosphate kinase (Coe E L, Greenhouse W V V. Biochimica et Biophysica Acta (BBA)—General Subjects 1973; 329:171-82), and thereby inhibit glucose uptake. Supporting evidence was also found for the PPP estimate. Treatment of Krebs ascites cells with (80 mM) oxamate dramatically reduced the activity of the PPP (by 50%) via a redox-coupled mechanism (Gumaa K A, McLean P. Biochem Biophys Res Commun 1969; 35:86-93). In this study, metabolic flux analysis calculations showed that the PPP flux in oxamate treated adipocytes was less than in untreated controls (by 82%). As adipocyte PPP activity is regulated by NADPH-consuming pathways (Fabregat I, Revilla E, Machado A. Mol Cell Biochem 1987; 74:77-81), the decrease in PPP flux may be due to a reduction in de novo fatty acid synthesis.

Accordingly, we showed that inhibiting lactate dehydrogenase or pyruvate carboxylase decreased triglyceride accumulation. Flux calculations revealed that both inhibitions down-regulated de novo fatty acid synthesis, citrate efflux from the mitochondria, and anaplerosis (PC flux). Under standard culture conditions with glucose as the main carbon source, 3T3-L1 adipocytes form triglyceride mainly through de novo fatty acid synthesis. De novo fatty acid synthesis requires export of citrate from mitochondria into cytosol, where it is cleaved by the ATP-dependent citrate lyase into oxaloacetate and the fatty acid chain precursor acetyl-CoA. This drain on a TCA cycle intermediate needs to be compensated by replenishing the oxaloacetate pool, which is an anaplerotic function attributed to pyruvate carboxylase. In this study, pyruvate carboxylase flux was reduced by both oxamate and phenylacetate treatments, but the underlying mechanisms could be different. The metabolism of phenylacetate produces phenylacetyl-CoA, which allosterically represses the activity of the pyruvate carboxylase enzyme. The effect of oxamate on pyruvate carboxylase likely reflected the decrease in glycolysis flux and the resulting limitation in pyruvate availability.

Another shared effect of both inhibitors was to up-regulate glycerol output, which is a direct indicator of lipolysis. As oxygen uptake rate was either not affected or decreased, the increased lipolysis unlikely reflected a higher demand for free fatty acids by β-oxidation. In adipocytes, lipolysis is regulated by several enzymes, including hormone-sensitive lipase (HSL), which catalyzes the hydrolysis of intracellular triglyceride and diacylglycerol (Carmen G Y, Victor S M. Cell Signal 2006; 18:401-8). Previous studies have shown that long-chain acyl CoAs (LC-CoAs) inhibit HSL activity (Hu L, Deeney J T, Nolan C J, et al. American journal of physiology 2005; 289:E1085-92; Jepson C A, Yeaman S J. FEBS Lett 1992; 310:197-200; Severson D L, Hurley B. Lipids 1984; 19:134-8). Thus, while not wishing to be bound by theory, one explanation for the up-regulation in lipolysis is that the down-regulation in de novo fatty acid synthesis (FIG. 3) lowered the LC-CoA levels, relieving the inhibition of hormone sensitive lipase. Consistent with this explanation, the inhibitor treatments significantly lowered the intracellular free fatty acids concentration (FIG. 2A).

Despite increasing lipolysis, the inhibitors did not trigger significant free fatty acid release into the medium (FIG. 2B), pointing to elevated reesterification rates in the treated adipocytes. Results of MFA showed that the retention of free fatty acids by reesterification reflects an increased availability of the glycerol moiety for triglyceride synthesis. Model calculations showed that the inhibitors raised the net flux of glycerol-3-phosphate (G3P) formation by 64˜75% (Supplementary Table 2, net difference between reactions #3 and #4). The underlying mechanisms could be different for oxamate and phenylacetate treatments. Inhibiting lactate dehydrogenase with oxamate increases the cytosolic NADH:NAD⁺ ratio, favoring the reduction of dihydroxyacetone phosphate into glycerol 3-phosphate by glycerol 3-phosphate dehydrogenase. In adipocytes treated with phenylacetate, inhibiting pyruvate carboxylase up-regulated the flux through glycolysis, which then can increase the availability of dihydroxyacetone phosphate.

In conclusion, the results of this study show that the pyruvate reactions exert significant flux control over triglyceride accumulation in adipocytes. These results demonstrate a novel strategy for intervention against adiposity in humans at the level of cellular metabolism, as the pathways of intermediary metabolism are extremely well conserved across different species.

1. A method of treating obesity, the method comprising: administering at least one agent that perturbs flux distribution of pyruvate to an obese individual with a BMI greater than 30 in an amount that results in a decrease in triglyceride storage in an adipocyte in the obese individual.

2. The method of claim 1, wherein the at least one agent shifts the flux of pyruvate from anaerobic glycolysis to the citric acid cycle.

3. The method of claim 1, wherein the at least one agent is selected from the group consisting of: an RNA interference molecule, a small molecule, a protein, a polypeptide, an antibody, an antibody fragment or a metabolite analog.

4. The method of claim 1, wherein the at least one agent inhibits at least one enzyme or enzyme complex selected from the group consisting of: lactate dehydrogenase, pyruvate dehydrogenase, pyruvate carboxylase, or any combination thereof.

5. The method of claim 3, wherein the at least one agent comprises an inhibitor of pyruvate carboxylase.

6. The method of claim 3, wherein the small molecule is oxamate, or phenylacetate.

7. The method of claim 3, wherein the RNA interference molecule comprises siRNA.

8. The method of claim 1, wherein at least two agents are administered to the obese individual.

9. A method of modifying triglyceride storage in an adipocyte, the method comprising contacting an adipocyte with at least one agent that perturbs flux of pyruvate, wherein the agent decreases storage of triglyceride in the adipocyte.

10. The method of claim 9, wherein the at least one agent shifts the flux of pyruvate from anaerobic glycolysis to the citric acid cycle.

11. The method of claim 9, wherein the at least one agent is selected from the group consisting of: an RNA interference molecule, a small molecule, a protein, a polypeptide, an antibody, an antibody fragment or a metabolite analog.

12. The method of claim 10, wherein the at least one agent inhibits at least one enzyme or enzyme complex selected from the group consisting of: lactate dehydrogenase, pyruvate dehydrogenase, pyruvate carboxylase, or any combination thereof.

13. The method of claim 12, wherein the at least one agent comprises an inhibitor of pyruvate carboxylase.

14. The method of claim 11, wherein the small molecule is oxamate or phenylacetate.

15. The method of claim 11, wherein the RNA interference molecule comprises siRNA.

16. The method of claim 9, wherein the adipocyte is contacted with at least two agents that perturb pyruvate flux.

17. A method of modifying triglyceride storage in an adipocyte, the method comprising contacting an adipocyte with at least one inhibitor of pyruvate carboxylase, wherein the inhibitor of pyruvate carboxylase decreases storage of triglyceride in an adipocyte.

18. The method of claim 17, wherein the inhibitor shifts the flux of pyruvate from anaerobic glycolysis to the citric acid cycle.

19. The method of claim 17, wherein the inhibitor is selected from the group consisting of: an RNA interference molecule, a small molecule, a protein, a polypeptide, an antibody, an antibody fragment or a metabolite analog.

20. The method of claim 19, wherein the small molecule is phenylacetate.

21. The method of claim 19, wherein the RNA interference molecule comprises siRNA.

22. A method of reducing the number of adipocytes or preventing increase in the number of adipocytes in an individual, the method comprising administering at least one agent that perturbs flux distribution of pyruvate to an individual undergoing a weight loss diet or to an individual who has reduced their body mass index by at least one point, wherein the at least one agent permits a reduction of adipocyte number or prevents an increase in the number of adipocytes in the individual.

23. A method for screening an agent that decreases triglyceride storage in a cell, the method comprising:
(a) contacting a cell with an agent that perturbs flux of pyruvate, and
(b) assessing triglyceride levels in the cell,
wherein if the triglyceride levels in the cell are lower than triglyceride levels in an untreated cell, then the agent decreases storage of triglyceride in the adipocyte.

24. The method of claim 23, wherein Oil Red O stain is used to assess triglyceride levels.

25. The method of claim 23, wherein the cell comprises an adipocyte or an adipocyte cell line.

26.-49. (canceled)
